(19) United States
(12) Patent Application Publication
(10) Pub. No.: US 2026/0355195 A1
(43) Pub. Date: Nov. 12, 2026

(54) METHODS FOR DETERMINING THE NATURAL TIME COURSE OF IMMUNE RESPONSE AND METHODS AND COMPOSITIONS FOR MODIFYING INTRACELLULAR PH FOR DISEASE CONTROL

(71) Applicant: Therazwimm Corporation, Huntington, WV (US)

(72) Inventor: Wei-Ping Zeng, Huntington, WV (US)

(73) Assignee: Therazwimm Corporation, Huntington, WV (US)

(21) Appl. No.: 19/121,739

(22) PCT Filed: Oct. 17, 2023
(86) PCT No.: PCT/US2023/077093

§ 371 Date: Apr. 16, 2025

Related U.S. Application Data
(60) Provisional application No. 63/416,614, filed on Oct. 17, 2022.
(60) Provisional application No. 63/456,791, filed on Apr. 3, 2023.
(60) Provisional application No. 63/536,720, filed on Sep. 6, 2023.

(51) Int. Cl. A61K 31/506; A61K 31/192; A61K 31/365; A61K 31/501; A61K 45/06; A61P 31/10; A61P 35/00; G01N 1/30; G01N 33/50; G01N 33/575; G01N 33/84

(52) U.S. Cl. CPC A61K 31/506; A61K 31/192; A61K 31/365; A61K 31/501; A61K 45/06; A61P 31/10; A61P 35/00; G01N 1/30; G01N 33/5011; G01N 33/5014; G01N 33/5759; G01N 33/57595; G01N 33/84; G01N 2001/302; G01N 2333/46; G01N 2500/10

(57) ABSTRACT

This invention provides methods to determine the stage in the natural time course of immune response in a subject. It describes method to control immune response by targeting regulatory mechanisms differentially operating in lymphocytes at different stages in the natural time course of immune response. One kind of such regulatory mechanism is the mechanisms of energy metabolism that control intracellular pH (pHi), which in turn control cell death and proliferation. This invention also describes a combinatorial approach to lowering pHi of neoplastic cells to induce their death using compositions of combinations of regulators of energy metabolism. Finally, this invention provides methods to treat skin or mucosa infections or neoplasia by topical application of pH modifier(s) that lower pH.

### RELATED APPLICATIONS

This application is a national stage filing under 35 U.S.C. § 371 of International Patent Application No. PCT/US2023/077093, filed Oct. 17, 2023, which claims the benefit under 35 U.S.C. § 119 (e) to U.S. Provisional Application No. 63/416,614, filed on Oct. 17, 2022; U.S. Provisional Application No. 63/456,791, filed on Apr. 3, 2023; and U.S. Provisional Application No. 63/536,720, filed on Sep. 6, 2023, the entire contents of each of which are incorporated herein by reference.

### BACKGROUND

New therapeutics and strategies to achieve high therapeutic efficacy and minimize adverse side effects benefit patients of a broad variety of diseases such as immunological diseases and cancers whose pathogenesis is attributable in part or in full to cell death and/or proliferation.

### SUMMARY

This invention describes a method or composition for controlling immune response via targeting an immune regulatory mechanism operating in lymphocytes at different stages in the natural time course of the immune response, wherein the targeting is guided and the natural time course is determined by the profiles of intracellular pH (pHi) and mitochondrial membrane potentials (MMP) of the lymphocytes.

In some embodiments, the immune regulatory mechanism comprises mechanisms of energy metabolism that impact the pHi of the lymphocytes.

In some embodiments, the stages in the natural time course of an immune response are determined by dividing the lymphocytes into populations of relatively high and low pHi that are referred to as the P and N lymphocytes or populations, respectively, and further by the profiles of both the pHi and the MMP of the P and N lymphocytes such that an early stage is determined by a predominance of N lymphocytes, and all lymphocytes show an inverse relation between MMP and pHi; an intermediate stage is determined by the appearance of P lymphocytes with a positive relation between MMP and pHi and the N lymphocytes may or may not remain as the dominant lymphocytes; a late stage is determined by a predominance of P lymphocytes with a positive relation between MMP and pHi.

At the late stage lymphocytes can be in one of four distinct energetic states: e1 state is characterized by the presence of substantial number of what are referred as the “n lymphocytes” in this application that are N lymphocytes that have low MMP and weak or no inverse relation between MMP and pHi, and the remaining N and P lymphocytes also have relatively low MMP; e2 state is characterized by the presence of small numbers of n lymphocytes and overall the rest of the lymphocytes have higher MMP than lymphocytes in the e1 state; e3 state is characterized by the lack of n lymphocytes, and strong positive and negative relation between MMP and pHi; e4 state is characterized by the predominance of n lymphocytes within the N population, only few if any N lymphocytes showing high MMP and inverse relation between MMP and pHi. Alternatively, the criteria for determining the stages in the natural time course of an immune response are the differential patterns of molecules and/or the activities of the molecules that are present or expressed in the lymphocytes at the different stages and energetic states as determined by the foregoing method. The method for determining the natural time course of immune response or the profiles of pHi and MMP of the lymphocytes comprises a laboratory technique to stain cells for surface molecule(s), pHi, MMP and Annexin V in a single step.

In some embodiments, the method is for treating and/or preventing an immunological disease.

In some embodiments, apoptosis is induced by lowering intracellular pH (pHi) whereas promoting cell survival and proliferation by raising pHi by modulating energy metabolism of the cell for disease treatment or prevention. In some embodiments, lowering pHi is achieved by increasing the influx of pyruvates and/or fatty acids to the TCA cycle, inhibiting amino acid energy metabolism, inhibiting glycolysis 17 for energy production, inducing imbalance between ATP synthesis and hydrolysis by restricting entry of carbon backbones of amino acids and/or influx of fatty acids to TCA cycle, or combinations thereof. In some embodiments, raising pHi is achieved by enhancing amino acid energy metabolism and/or glycolysis for energy production, restricting influx of pyruvates and/or fatty acids to the TCA cycle, restoring balance between ATP synthesis and hydrolysis, or combinations thereof.

In some embodiments, the immunological diseases are those with pathogenesis attributed partially or fully to overzealous immune response. They comprise but are not limited to autoimmune diseases, allergic diseases, and infectious diseases where immune responses impair the structure or functions of host tissue or organ. The treatment of such disease comprises lowering pHi of lymphocytes to induce apoptosis according to the natural time course of the immune response or the profiles of pHi and MMP of the lymphocytes or the relative proportions of P and N lymphocytes: at the early stage primarily by increasing the influx of pyruvates and/or fatty acids to the TCA cycle, and by inhibiting amino acid energy metabolism and/or glycolysis for energy metabolism, or combinations thereof; at the intermediate stage primarily by inhibiting amino acid energy metabolism, increasing the influx of pyruvates and/or fatty acids to the TCA cycle, and optionally also by inhibiting glycolysis for energy production, inducing imbalance between ATP synthesis and hydrolysis by restricting entry of carbon backbones of amino acids and/or influx of fatty acids to TCA cycle, or combinations thereof; at the late stage in the natural time course of immune response primarily by inducing imbalance between ATP synthesis and hydrolysis by restricting entry of carbon backbones of amino acids and/or influx of fatty acids to TCA cycle, inhibiting glycolysis for energy production and/or amino acid energy metabolism, and optionally by increasing influx of pyruvates and/or fatty acids to TCA cycle, or combinations thereof.

In some embodiments, the immunological diseases are diseases whose pathogenesis is partially or fully attributed to insufficient immune response to infection, vaccine or cancer, and wherein the diseases are treated or prevented by raising pHi of lymphocytes to promote survival and proliferation of the lymphocytes.

In some embodiments method or composition are for lowering intracellular pH (pHi) to induce apoptosis of neoplastic cells, wherein the method comprises: creating a panel of compositions of individual regulators of energy metabolism at low doses or concentrations and combinations of the individual regulators to lower pHi of the neoplastic cells; and screening such panel of compositions for inhibition of the growth and/or survival of the neoplastic cells; and with or without simultaneously screening such panel of compositions for inhibition of the growth or survival of normal cells; and selecting composition(s) that show relatively strong inhibition of neoplastic cells, with or without also considering relatively weak or no inhibition of normal cells, and further adjusting the dose(s) of one or more regulator(s) of energy metabolism in the selected compositions to achieve optimal inhibition of neoplastic cells but minimize inhibition of normal cells. The regulators of energy metabolism of the panel of compositions are selected from those that increase the influx of pyruvates and/or fatty acids to TCA cycle, inhibit amino acid energy metabolism, inhibit glycolysis for energy production, or induce imbalance between ATP synthesis and hydrolysis by restricting entry of carbon backbones of amino acids and/or influx of fatty acids to TCA cycle. In some embodiments, the panel of compositions comprise compositions summarized in Table 1 in this application and compositions of metabolic regulators with similar or identical activities. In some embodiments, compositions comprising regulators of energy metabolism identical or similar to those of compositions 8′, 12′ 15, 16′, 19, 21′, 8″, 12″ and 16″ described in Table 1 and Example K are potential anti-cancer drugs.

In some embodiments, Ki-67 is used as a biomarker to predict a subject's responsiveness to low pHi-based cancer therapy wherein the level of Ki-67 expression in cancer cells or the percentage of cancer cells expressing high level(s) of Ki-67 serves as a predictor for whether the subject would respond to cancer therapy with pH modifier(s) that lower pHi.

In some embodiments, skin or mucosa infection(s) or neoplasia are treated by topical application of pH modifier(s) that lower pH.

Further provided herein are methods or compositions for inducing apoptosis by lowering intracellular pH (pHi) or promoting cell survival and proliferation by raising pHi, by modulating energy metabolism of the cell for disease treatment or prevention, whereby,
lowering pHi is achieved by increasing the influx of pyruvates and/or fatty acids to the TCA cycle, inhibiting amino acid energy metabolism, inhibiting glycolysis for energy production, inducing imbalance between ATP synthesis and hydrolysis by restricting entry of carbon backbones of amino acids and/or influx of fatty acids to TCA cycle, or combinations thereof;
raising pHi is achieved by enhancing amino acid energy metabolism and/or glycolysis for energy production, restricting influx of pyruvates and/or fatty acids to the TCA cycle, restoring balance between 9 ATP synthesis and hydrolysis, or combinations thereof; and
modulating energy metabolism is achieved by administering a composition comprising regulator(s) of energy metabolism.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiments disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1D show time progression of immune response in P (high pHi) and N (low pHi) populations of the indicated live lymphocyte populations in the MLNs of mice after challenges with OVA or in the DLNs of mice after immunization with MOG. FIGS. 1A-1C show early (FIG. 1A), intermediate (FIG. 1B), and late (FIG. 1C) stages in the natural time course of immune responses to OVA challenges. FIG. 1D show an example of the stage in the natural time course of immune response to MOG immunization. Lym: total live lymphocytes, CD4: live CD4 T cells, CD8: live CD8 T cells, CD19: live B cells.

FIGS. 2A-2E show inhibition of lymphocytes in the MLNs of OVA-challenged mice at the intermediate stage in the natural time course of immune response by metabolic regulators. FIG. 2A shows that the pHi in the different live lymphocyte populations were lowered by treatment of the mice with inhibitor that inhibits glycolysis (GSK) or glutaminolysis (CB-839) (the upper panels), or promoter of the influx of pyruvates (DCA) or fatty acids (C75) to the TCA cycle (lower panels). FIG. 2B is a set of flow cytometry dot plots showing the profiles of pHi and MMP, and the P and N populations of the same lymphocytes in FIG. 2A. FIG. 2C are a set of bar graphs for numerical presentation of the indicated data (% of P and N lymphocytes, fold changes of absolute live cell numbers, and the mean fluorescence intensities of pHrodo™ Green staining) in FIG. 2B. FIG. 2D is a set of dot plots early apoptotic cells (Annexin V⁺) in the indicated live lymphocyte populations and treatment groups. FIG. 2E is a set of bar graphs showing the percentages of early apoptotic cells from data in FIG. 2D, and fold changes of the absolute live cell numbers in the indicated lymphocyte populations, and treatment groups.

FIGS. 3A-3C show inhibition of lymphocytes in the MLNs of OVA-challenged mice at the early stage in the natural time course of immune response by metabolic regulators. FIG. 3A is a set of flow cytometry dot plots showing the profiles of pHi and MMP, and the P and N populations of lymphocytes derived from mice treated with vehicle control, CB-839 or DCA. FIG. 3B is a set of dot plots early apoptotic cells (Annexin V.) in the indicated live lymphocyte populations and treatment groups. FIG. 3C is a set of bar graphs for numerical presentation of data in FIG. 2B, showing the percentages of early apoptotic pHi-18 low cells, and the pHi of the N lymphocytes.

FIGS. 4A-4E show inhibition of lymphocytes in the MLNs of OVA-challenged mice in the late-e1 state in the natural time course of immune response by metabolic regulators. FIG. 4A is a set of flow cytometry dot plots showing the profiles of pHi and MMP, the P and N populations of lymphocytes derived from mice treated with the different metabolic regulators as indicated. The n populations within the N lymphocytes that had low MMP and weak or no correlation between MMP and pHi. CPI-613 is a blocker of the entry of the carbon backbones of amino acids to the TCA cycle. Etomoxir (Etom) is an inhibitor of the influx of fatty acid to the TCA cycle. FIG. 4B is a set of bar graphs for numerical presentation of data in FIG. 4A, showing the percentages of P and N lymphocytes, and fold changes of their absolute numbers, and such changes in the indicated total live lymphocyte populations. FIG. 4C is a set of dot plots early apoptotic cells (Annexin V.) in the indicated live lymphocyte populations and treatment groups. FIG. 4D are bar graphs showing percentages of early apoptotic cells (upper) and pHi of the N lymphocytes (lower) in the indicated lymphocyte populations and treatment groups. FIG. 4E is a set of dot plots comparing the profiles of pHi and MMP of total live lymphocytes in the indicated treatment groups.

FIGS. 5A-5B show the lowering imbalance between ATP synthesis and hydrolysis in lymphocytes in the late-e2 state. FIG. 5A is a bar graph showing the relative intracellular ATP counts in live lymphocytes in the MLNs of mice in the indicated treatment groups. FIG. 5B shows intracellular ATP and Ki-67 expression in sorted lymphocyte populations. The left panel shows the sorting gates.

FIGS. 6A-6B show inhibition of lymphocytes in the MLNs of OVA-challenged mice in the late-e1 state in the natural time course of immune response by metabolic regulators. FIG. 6A is a set of bar graphs showing the fold changes of absolute live cell numbers and percentages of early apoptotic cells in the indicated live lymphocyte populations and treatment groups. FIG. 6B is a set of dot plots comparing the profiles of pHi and MMP (upper panels) and early apoptotic cells (lower panels) in total live lymphocytes of mice treated with vehicle or DCA. The Numbers in large font indicate the percentages of N lymphocytes and early apoptotic pHi-low cells.

FIG. 7 shows inhibition of live lymphocytes in the MLNs of OVA-challenged mice in the late-e3 state in the natural time course of immune response by metabolic regulators. The upper and lower 13 panels show fold changes of absolute live cell numbers in the indicated lymphocyte populations and treatment groups.

FIG. 8 shows inhibition of live lymphocytes in the MLNs of OVA-challenged mice in the late-e4 state in the natural time course of immune response by metabolic regulators. Shown are bar graphs of the fold changes of absolute cell numbers, pHi, and percentages of early apoptotic cells in the indicated 18 lymphocyte populations and treatment groups.

FIG. 9A-9B shows inhibition of immune response in mice that received 2 rounds of OVA challenges by metabolic regulators. FIG. 9A are bar graphs of the fold changes of absolute cell numbers in the indicated lymphocyte populations in the MLNs and treatment regimens described in Example I. FIG. 9B shows the airway resistance of mice in the indicated treatment groups.

FIGS. 10A-10B show inhibition of live lymphocytes of by metabolic regulators in the DLNs and NDLNs of mice immunized with MOG peptide in the EAE model. FIG. 10A are two sets of dot plots showing the pHi and MMP profiles (left panels) and early apoptotic cells (right panels) of live total lymphocytes or CD4 T cells in the DLNs or NDLNs of the mice in the indicated treatment groups. FIG. 10B is a set of bar graphs showing the percentages of N lymphocytes, pHi and percentages of early apoptotic cells in the indicated live lymphocyte populations and treatment groups.

FIGS. 11A-11D show the screening of compositions of metabolic regulators for cancer therapy by the combinatorial approach.

FIG. 12 shows the correlation between Ki-67 levels and susceptibility Raji tumor cells to low pHi-induced death. The top left panel shows that the pHi of the Raji cells was lowered by treatment with HOAc but not NaOH. The top right panel shows that early apoptosis occurred in the pHi-low Raji cells. The lower left panel is overlaid histograms shows that different subsets of Raji cells expressed high, low, or low to negative levels of Ki-67. Mean fluorescence intensities (MFI) of Ki-67 in these subsets. The lower right panel shows the fold of live cells of the different subsets of Raji cells after treatment with HOAc relative to treatment with saline.

FIGS. 13A-13C show the treatment of skin or mucosa infections with HOAc. FIG. 13A is a copy of medical statement showing the clinical diagnosis of onychomycosis on the great toe in FIG. 13B left. FIG. 13B are photographs of the toenail affected by onychomycosis before and after 1.5 months of treatment with compositions comprising HOAc in water. FIG. 13C are photographs showing two lesions of infection 9 (indicated by arrows) on the mucosa of the upper lip before treatment (DO) and one (D1) and two days (D3) after treatments with a composition comprising HOAc in water.

FIG. 14 is a set of two photographs showing two lesions of benign neoplasia (indicated by arrows) of facial skin before (D0) and after 20 days of treatment with compositions comprising HOAc in water.

### DEFINITIONS

Unless defined otherwise, all technical and scientific terms used herein have the same meaning as commonly understood to one of ordinary skill in the art to which the presently disclosed subject matter pertains. Following long-standing patent law convention, the terms “a”, “an”, and “the” refer to “one or more” when used in the subject specification, including the claims. Thus, for example, reference to “a cell” can include a plurality of such cell, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used in this invention, the term “about”, when referring to a value or to an amount of mass, weight, time, volume, concentration, dose, and/or percentage can encompass variations of, in some embodiments+/−0.01-50% from the specified amount, as such variations are appropriate in the disclosed packages and methods.

The terms “administration,” “administer,” or “administering” as used in this invention refer to the delivery of a composition into or on the body of a subject, either systemically or locally to a specific anatomical location via any of a variety of routes and means of administration. Suitable routes and means of administration include (but are not limited to) intravenous (i.v.) injection/perfusion, intra-peritoneal (i.p.) injection, intratracheal (i.t.) injection, instillation or spray, inhalation of aerosols or vapors, topical application, intranasal spray, intradermal injection, subcutaneous injection, and rectal application. The means of administration also include the velocity of administration, i.e., the amount of composition delivered to a subject in a unit of time. Administration to a specific anatomical location may be carried out with or without assistance with guiding/navigation technologies such as imaging technologies. The selection of pharmaceutically acceptable routes and means of administration depends on the nature of the disease, the tissue(s) and/or organs affected by the disease, and should be determined by a subject's attending health care provider(s) within the scope of sound medical judgment.

“Allergic disease” as used in this invention refers to diseases caused by inflammatory and/or immune responses to allergen exposure.

The term “anatomical location” refers to any spatial point inside or on the body of a subject. For example, an anatomical location can be a location in the interstitial space of a tissue, the lumen of an organ, a body cavity, the space inside the blood vessels, an area of the mucosa, or skin.

The term “aqueous environment” refers to extracellular and/or intracellular fluid at any anatomical location within or on a subject, intracellular fluid of in vitro cultured cells, cell culture medium, and any aqueous solutions prepared in vitro. Examples of extracellular fluids include (but are not limited to) interstitial fluid, plasma, lymphatic fluid, mucus, bronchoalveolar fluid, cerebrospinal fluid, synovial fluid, fluids of chest and abdominal cavities.

The terms “as example(s)”, “for example” and “such as” as used in this invention, are not limiting, and do not exclude other subject matters similar to the example(s).

“Autoimmune disease” as used in this invention refers to diseases that are characterized by immune and inflammatory responses to one or more components of a subject's own tissues, cells, and/or bodily fluids.

“Composition” as used in this invention refers to a pharmaceutical formulation comprising certain amounts of one or more pharmaceutically accepted compounds with or without one or more other medicines formulated with one or more pharmaceutically acceptable solvents, solutions, excipients, and/or carriers. While maintaining the therapeutic or preventive efficacy of a composition, the concentration(s) of pH modifier(s) and/or cell proliferation inhibitor(s) must be set at such levels that the composition meets the standard of “pharmaceutically acceptable”. A composition may be in any of a variety of physical forms, including (but not limited to) liquid, aerosol, vapor, cream, gel, capsule, tablet, powder, granules, and the like. All or some of the components of a composition may be pre-mixed or supplied separately and mixed before use. It will be understood that the composition to be used on a given subject at a given time will be decided by the subject's attending health care provider(s) within the scope of sound medical judgment.

“Immune response” as used in this invention refers to the activation, proliferation, migration and functional execution of innate, adaptive, or both innate and adaptive immune cells in specialized lymphoid organs/tissues such as the lymph nodes, spleen and Peyer's patches, and the anatomical location of antigen exposure; and the cellular and molecular consequences thereof. Innate immune cells are activated by receptors other than antigen receptor. The activation of the adaptive immune cells (mainly B and T lymphocytes and NKT cells) primarily results from the engagement of antigen receptors (BCR or TCR) with antigens or superantigens but may also be caused by stimulation with B or T cell mitogens (e.g., Lipopolysaccharides, Concanavalin A), and also includes by-stander activation by cytokines.

The term “infectious disease” refers to a disease that can be transmitted from one subject to another, and is caused by microbial agent(s) (e.g., pneumonia). The microbial agents can be bacteria, viruses, fungi or parasites. Not only the microbial agents themselves can directly harm the infected subject, overzealous inflammatory and/or immune responses to the microbial agents can also cause pathology and pathophysiology, whereas adequate inflammatory and/or immune responses to the microbes are necessary for the clearance of the infection.

The terms “Inflammatory cells” and “immune cells” as used in this invention are interchangeable and refer to both the innate and adaptive immune cells and include cells of the lymphoid and myeloid lineages. Inflammatory cells may circulate through the blood and lymphatic systems and may migrate to and take residence in specific tissues or anatomical locations.

“Inflammatory disease” and “immunological disease” as used in this invention are interchangeable and refer to a disease or condition, in which inflammatory and/or immune responses or the lack or insufficiency thereof play a pathogenic role.

The term “inhibitor” refers to a molecule or a mixture of different molecules that exerts the effect of alteration, interference, reduction, down-regulation, blocking, suppression, abrogation, or degradation (directly or indirectly) of the expression, amount, or activity of an enzyme, membrane transporter or ion channel or any other molecules that play a role in a biochemical or biological process.

“Neoplasia” or “neoplastic” are used in this invention to describe dysregulated, benign or malignant cell proliferation. Solid and nonsolid tumors are neoplastic diseases. Malignant solid and nonsolid tumors are synonymous with cancers.

The term “normal cell” as used in this invention refers to a cell that does not play a pathogenic or adverse role in a disease. A normal cell may be a cell that plays a protective or preventive role against a disease, or a cell irrelevant to the disease.

“Overzealous” is used in this invention to characterize inflammatory and/or immune responses that impair the structure and/or function of the tissue(s) and/or organ(s) of a subject. Immune and/or inflammatory responses to allergens and self-antigens in allergic and autoimmune diseases, respectively, are generally considered as overzealous immune and/or inflammatory responses. Immune and/or inflammatory responses to infection can also be overzealous when such responses damage normal tissue or organs.

“pH modifier” as used in this invention refers to any pharmaceutically acceptable compound that can alter, or help resist the change of, the pH of an aqueous environment.

The term “pharmaceutically acceptable” refers to the characteristics of any molecule and/or compound or a mixture of different molecules or compounds that, within the scope of sound medical judgment, is suitable for use in a subject without causing excessive toxicity, allergic response, irritation, and/or other problem or complication, commensurate with reasonable risk/benefit ratio.

“Proliferation” as used in this invention refers to the increase of the number of cells by cell division and/or the survival of the dividing cells.

The term “subject” as used in this invention refers to a human being or non-human animal (e.g., mouse, dog, cat, cattle, horse). A subject seeking diagnosis, treatment, and/or prevention of a disease is referred to as a patient.

“Activity”, or “activities”, of a molecule, refers to the biochemical or biological consequence(s) when a molecule is introduced into or present in a cell or an extracellular environment.

“Amino acid energy metabolism”, as used in this application, refers to any of the biochemical reactions and molecular events that lead to the breakdown of arginine, glutamine, histidine, or proline to produce ammonia or urea and glutamate; the export of glutamate out of the cell; or alternatively the participation of glutamate in the malate aspartate shuttle; or further metabolism of the glutamate to produce malate and aspartate to participate in the malate aspartate shuttle; and subsequent catabolic processes or reactions that further convert malate to lactate and the secretion of lactate to extracellular space; when this term is used with other amino acids, it specifically refers to the reactions that remove the amine groups from the amino acids to produce ammonia. The term ammonia as used in this definition encompasses its conversion to ammonium and vice versa so that the nitrogen atoms derived from the amine groups of the amino acids may exist in the equilibrium between ammonia and ammonium. Glutaminolysis is a specific type of amino acid energy metabolism that catabolizes glutamine.

“Differentially expressed or present”, as used to describe molecules or part(s) of the molecules on or in a lymphocyte, refers to qualitative (present or not) or quantitative differences of one or more molecules or part(s) of the molecule(s) or their activities at the early, intermediate and late stages in the natural time course of immune response, or alternatively in different lymphocytes distinguishable based on their profiles of pHi and MMP.

“Entry of carbon backbones of amino acids to TCA cycle”, refers to the step of biochemical reactions that converts amino acids to alpha-ketoglutarate, succinyl-CoA, fumarate, or oxaloacetate and the immediate catabolic reactions of these metabolites in the TCA cycle, for example the reaction catalyzed by alpha-ketoglutarate dehydrogenase in the TCA cycle; and the step of biochemical reactions that convert amino acids to pyruvate.

“Glycolysis for energy production” or “glycolysis” for short, as used in this application, refers to the import of glucose into a cell and the subsequent biochemical reactions in the cytosol that net produce 2 ATPs and 2 lactates per glucose molecule; and the excretion of the lactates out of the cell.

“Influx of fatty acids to (the) TCA cycle”, refers to the processes that transport fatty acid into the cell, convert the fatty acid to fatty acyl-CoA, transport the fatty acyl-CoA to mitochondria, and beta oxidation that breaks down the long carbon chain of the fatty acyl CoA to generate acetyl COA. Activators and inhibitors of the activities or expression of the proteins or enzymes that participate or regulate these processes, particularly but not exclusively those in the rate-limiting step, for example the carnitine palmitoyltransferase 1 (CPT-1), are part of the regulators of energy metabolism in this disclosure.

“Influx of pyruvates to (the) TCA cycle”, as used in this application, refers to the processes that import pyruvate into the mitochondria, catabolize it to produce acetyl COA, or directly convert pyruvate to oxaloacetate, to participate in the TCA cycle; as well as the processes that regulate these events, for example the phosphorylation of pyruvate dehydrogenase (PDH) by PDH kinase that inactivates the PDH. The inhibitors and activators of the activities or expression of the proteins or enzymes in these processes are part of the regulators of energy metabolism in this disclosure.

“Low dose”, as used to describe the regulator(s) of energy metabolism, refers to the dose of a regulator of energy metabolism that when administered to a subject alone or in combination with other regulator(s) of energy metabolism or other compound(s) does not cause adverse side effect(s) that are more severe than what are generally considered to be acceptable by the medical community.

“Lymphocytes”, as used in this application, can be all lymphocytes, or subpopulation(s) of lymphocytes that are relevant to the pathogenesis of a specific disease, for example, CD4 T cells in multiple sclerosis and its animal model experimental autoimmune encephalomyelitis (EAE), or B cells in lupus. Normal lymphocytes are lymphocytes derived from a subject or healthy donor(s) that are not neoplastic.

“Mitochondrial membrane potential”, refers to the electrical potential across the mitochondrial inner membrane that is created by the proton gradient between the mitochondrial matrix and the inter-membrane space. The mitochondrial membrane potential can be measured by various methods, for example, by staining cells with the MitoSpy fluorescence dyes (Biolegend) followed by flow cytometric detection of the fluorescence in the cells.

“Regulator of energy metabolism” and “metabolic regulator” are used interchangeably in this disclosure, refer to an activator or inhibitor of the activity or expression of a protein or enzyme that participates or regulates the biochemical reactions that catabolize glucose, fatty acids and amino acids to produce ATP. The term activator, stimulator, accelerator and promoter are used interchangeably in this sense.

“Significantly expressed”, as used to described the expression of a protein or the gene of the protein, refers to the showing of specific positive signals of detection as compared with negative control, for example, positive signal in flow cytometry or Western blotting using specific but not nonspecific antibody against the protein for detection, or RT-PCR using specific but not nonspecific primers for the gene of the protein. In some cases, in order to select effective protein or enzyme target(s) for a particular type of cancer, the expression of the protein or enzyme in such cancer may be compared against a panel of other cancers or normal tissues or cells such as normal lymphocytes, and relatively strong (not necessarily higher than the comparators) expression in the particular type of cancer will be the basis for selecting the protein or enzyme as a potential effective target.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter, for example, use of different compounds with similar or identical activities as those described in the embodiments and examples.

An immune response can be beneficial or detrimental to the health of a subject. Immune responses to infectious agents or cancer cells protect an individual, whereas immune responses to allergens or self-antigens cause allergic and autoimmune diseases, respectively. In fact, even immune responses to infection, for example in Leishmaniasis, can also cause immunopathology in the host. Therefore, therapeutics that manipulates immune responses can benefit patients who suffer from a large variety of diseases.

An immune response is a dynamic process. A typical adaptive immune response to infection lasts 2-3 weeks, and ceases when the infection is cleared (1). The overall immune response to allergens or self-antigens where antigenic stimulation is repetitive may last for longer time, but the life cycle of an individual lymphocyte is similar. In this regard, lymphocytes in an immune response initially undergo clonal expansion or proliferation and, in the meantime, also undergo active cell death by apoptosis. Eventually most of the expanded lymphocytes die of apoptosis whereas a small numbers of them become memory cells. (21,22). Therefore, manipulating the death and proliferation of lymphocytes can not only control the magnitude of the ongoing immune response but also affect memory formation by impacting on the fate of precursors of the memory cells, which will determine the prospect of future response to the same antigen. The mechanisms of regulating lymphocyte death, proliferation, migration and functions may evolve along the natural time course of immune response. In order to effectively manipulate an immune response, therapeutics must target the mechanism operating in the lymphocytes at the time of medical intervention. However, this notion has not been well appreciated, by and large current therapeutics of immunological diseases do not take into consideration of kinetic changes of the immune regulation.

This could be due to the fact that most immunological studies are conducted in laboratory settings where the time of antigen exposure is known so that researchers know by experience when to collect samples for their specific study. In contrast, under clinical conditions, it is usually not known when a subject was first exposed to antigen or how long the immune response has been going on in a subject at the time the subject seeks medical treatment. Therefore, in order to intervene an immune response according to its natural time course, there must be a way to determine the natural time course of immune response or the stages in such time course. However, there is no such method currently available.

Energy metabolism plays an important role in immune response. Earlier studies found that like what is observed in tumor cells and known as the Warburg effect, proliferating lymphocytes have a tendency to use glycolysis for energy production. (23). More recently, studies have shown that the choice of energy metabolism also plays a role in lymphocyte subset differentiation (24). In tumor cells, the contribution of glycolysis to total energy production ranges from less than 5% to over 50% (2). The relative contribution of glycolysis to energy production in lymphocytes is not clear. Extensive studies in the past have also identified the PI3K/AKT/mTOR pathway as the mechanism for promoting the Warburg effect (25). Apart from the Warburg effect, glutaminolysis is another feature of highly proliferating cells, and considered another hallmark of cancer cells (3). In fact, 20% of cancer patients ultimately die of cachexia as a result of breaking down adipose tissues and muscle to replenish glutamines in the blood for consumption by cancer cells (4).

In the Warburg effect, the waste product of glycolysis lactic acid is excreted to extracellular space, which creates an acidic extracellular tumor microenvironment (TME). Studies of the biological consequence of the Warburg effect have been focused on such an acidic TME. It has been reported that the acidic TME can promote tumor cell invasion of local tissues (5). It also negatively regulates the functions of innate immune cells and cytotoxic T cell activities (6-10). However, little is known how the Warburg effect and glutaminolysis may regulate the fate as to the survival and rate of proliferation of the highly proliferating cells themselves. The salient feature of the Warburg effect is its low efficiency for energy production as it net produces only 2 ATPs as opposed to up to 34 ATP per glucose by mitochondrial respiration (11). Likewise, although glutamine is highly catabolized in tumor cells, at least half of its carbons are not oxidized to produce ATP but rather secreted as lactate by tumor cells (12). Thus, the tendency of proliferating cells to use the Warburg effect and glutaminolysis for energy metabolism creates a paradox between the high demand for energy by the proliferating cells and the low efficiency of energy production by these two pathways. This paradox, particularly how it may effect the fate of the proliferating cells, has not been explained. The inventor of the present inventions has found that low intracellular pH (pHi) induces apoptosis whereas high pHi allows the cells to proliferate at high rates in a previous pending patent application (International Application No. PCT/US22/47932, the entire contents of which is incorporated herein by reference). These findings were made in cells without any treatments therefore the cells maintained intact mitochondria, which distinguish them from earlier studies that described cytosolic acidification as a result of redistribution of mitochondrial contents due to damages to the mitochondria by experimental treatments (13-16). Data disclosed in this application further show that distinct metabolic pathways regulate pHi in distinct manners. Glutaminolysis and glycolysis for energy production play indispensable role in raising pHi or maintaining high pHi to avoid cell death that would be otherwise be induced by low pHi resulted from energy production by other pathways.

Given the importance of energy metabolism in immune response and tumorigenesis, it is an attractive idea to manipulate energy metabolism for treating immunological diseases and cancers. For immunological diseases, such efforts are still at a nascent stage. In contrast, there has been a rather long history of developing cancer therapeutics that target metabolic pathways. Unfortunately, this effort has not met with much success. The main obstacle is the dilemma between achieving therapeutic efficacy and avoiding adverse side effects (17). The pathway preferentially used in the cancer is usually chosen as the target. To achieve high therapeutic efficacy, such pathway must be blocked to near completion. However, the critical enzyme for such pathway, for example the lactate dehydrogenase for the Warburg effect, is often elevated in cancer cells (18). Therefore, in order to effectively block such pathway, high dose of inhibitor must be used. Since most metabolic pathways are shared by cancer and normal cells, high dose of inhibitor also causes severe adverse side effect. This application addresses this problem by employing a new strategy, i.e., to lower pHi of cancer cells by only partially manipulating, in some case by both partially blocking and partially enhancing, multiple pathways simultaneously to induce apoptosis of the cancer cells. Since no single pathway is blocked to near completion or driven over the edge, this strategy is less likely to cause severe side effect, but in the meantime the relatively small alterations of multiple pathways can collectively drive down the pHi enough to induce apoptosis of the cancer cells, as well as undesired pathological immune cells.

Pharmacological manipulation of energy metabolism can be considered as a special type of chemotherapy. For chemotherapeutics, as well as many other types of therapeutics, it is important to have a biomarker that can predict responsiveness of the cancer to the therapy. However, predictive biomarker for most of the currently available chemotherapeutics remains elusive (19). Ki-67 is a widely used, dependable marker for proliferating cells whose level positively correlates with the level of rRNA and DNA synthesis (20). However, Ki-67 is not a reliable predictive biomarker for existing chemotherapeutics (19).

In contrast, data disclosed in this application show that it could be a predictive biomarker for low pHi-based cancer therapy.

### Method for Determining the Natural Time Course of Immune Response

When a subject with an immunological disease is presented to a health care provider, it is often hard to determine exactly when lymphocytes in the subject were exposed to the pathological antigen (e.g., microbial product, allergen or autoantigen). As a result, it cannot be determined at what time point the immune response is when the subject seeks medical treatment. Knowing or determining the natural time course of an immune response is important for effectively modulating the immune response because, as disclosed herein, the mechanisms for energy generation in the lymphocytes are different at different time points in the course of an immune responses. Such differences are important for the control of death and proliferation of lymphocytes. Other mechanisms of controlling lymphocytes death, survival, proliferation, and functions may also differ when the lymphocytes are at early, intermediate or late stages of an immune response. Thus, a method that enables a health professional to determine the natural time course of an immune response can also be applied to other treatments targeting a variety of immune regulatory mechanisms to improve the targeting precision and efficacy. However, currently this problem has not been well appreciated and studied by the clinical and biomedical research communities, and there is no method available to determine the natural time course of immune response.

This disclosure offers such a method by examining the pHi and MMP of the lymphocytes. In this method, the lymphocytes are categorized into two populations, one with relatively high pHi and the other relatively low pHi, referred to as the P and N lymphocytes or populations, respectively. The early stage of an immune response is dominated by the N lymphocytes whereas the P lymphocytes are the minority, but all lymphocytes at this stage show an inverse relation between their MMP and pHi. The intermediate stage is marked by the appearance of P lymphocytes that have assumed a positive relation between their MMP and pHi, whereas the N lymphocytes continue to show an inverse relation between their pHi and MMP. In this stage the N lymphocytes may or may not remain as the dominant lymphocytes. As the immune response progresses further, the percentages of P lymphocytes increase and they maintain a positive relation between their MMP and pHi. When the P lymphocytes become the dominant lymphocytes, the immune response enters the late stage.

Lymphocytes at the late stage can be in any of four energetic states of late-e1, -e2, -e3 and -e4. The e1 state is characterized by the presence of substantial number of “n lymphocytes” that are N lymphocytes but have low MMP and weak or no inverse relation between MMP and pHi. Overall and the remaining N and P lymphocytes also have relatively low MMP. The e2 state is characterized by the presence of small numbers of n lymphocytes and overall the rest of the lymphocytes have higher MMP than lymphocytes in the e1 state. The e3 state is characterized by the lack of n lymphocytes, and strong positive and negative relation between MMP and pHi in the P and N lymphocytes, respectively. Finally, the e4 state is characterized by the predominance of n lymphocytes within the N population, only few if any N lymphocytes have high MMP and strong inverse relation between MMP and pHi.

However, in some instances, an immune response may not go through all the different stages. Depending on the history and degree of exposure to environment antigens, an immune response may start at a stage later than the early stage. In one embodiment with the EAE model, lymphocytes of unimmunized littermates showed profiles of MMP and pHi similar to those of the late-e2 state. This indicates that the mice had active ongoing immune response prior to immunization with specific antigen. Nonetheless, after immunization, the N population of the lymphocytes in the draining lymph nodes (DLNs) progressively declined even though they had skipped the early and intermediate stages. While the dynamic changes of the pHi and MMP of the lymphocytes do reflect the natural time course of an immune response, for practical purpose it is possible to design therapeutic strategy simply based on the profiles of pHi and MMP of the lymphocytes without employing the concept of natural time course of immune response. Such use of different narrative shall not be construed as material difference from this disclosure.

This disclosure extends the claim of the method to determine the natural time course of immune response to include the use of differential patterns of molecules and/or activities of the molecules that are present/expressed in lymphocytes at the different stages defined by their pHi and MMP profiles. This is a logical extension as it is obvious that the differential patterns of molecules and/or the activities of the molecules should have similar or equal power as the profiles of pHi and MMP in determining the stage in the natural time course of an immune response. This method may also be used in conjunction with other methods of defining different lymphocyte subpopulations, for example, one or more of methods for identifying antigen-specific lymphocytes using MHC tetramers, detecting surface markers of memory cells, etc. The lymphocytes in the method can be derived from tissues or body fluids, for example, lymph nodes, spleen, mucosa, blood, cerebrospinal fluid, bronchoalveolar lavage fluid, etc. While all lymphocytes may be analyzed, in cases where a particular subpopulation of lymphocytes is most relevant to the pathogenesis of a disease, for example, the CD4 T cells in multiple sclerosis and EAE, B cells in lupus, analyses may be directed such subpopulations of lymphocytes. In cases where MHC tetramers are available to detect antigen specific T cells, analyses may be focused on such T cells.

For convenient clinical application of the method, a kit containing all necessary reagents, for example, reagents for detecting pHi, MMP, molecules and/or their activities that are differentially present/expressed in lymphocytes at the early, intermediate and late stages of immune response, as well as detailed instruction of the method, will be provided to medical or veterinary professionals for determining the stage(s) of immune response(s) in a subject. Equipment and laboratory tools may also be supplied as a part of the package.

A Single-Step Technique for Staining Cell Surface Marker(s), pHi, MMP and Annexin V

In this disclosure, pHi, MMP and early apoptosis, i.e., positive staining of Annexin V, are detected by flow cytometry. Because different buffer and temperatures are used, the usual procedure for preparing cells for flow cytometry for this purpose would include at least four steps: staining cells for surface marker(s) at 4 °C, staining cells with a pH indicator at 37 °C, staining cells with MitoSpy at 37 °C to measure MMP, and finally staining cells in Annexin binding buffer at room temperature to detect early apoptotic cells. Such prolonged procedure is not only laborious, it also increase the risk of introducing artifacts, for example, cell death caused by multiple steps of incubating the cells at 37 °C or room temperature in serum-free buffers. To overcome these problems and achieve experimental results that best reflect in vivo immune response, a single-step technique was designed to stain cell surface marker(s), pHi, MMP and Annexin V all in a Ca²⁺-containing buffer for a short period of time at 37 °C. The Ca²⁺-containing buffer can be, but is not limited to, Annexin V binding buffer or Live Cell Image Solution (LCIS) (Life Technology, Grand Island, NY) with or without Ca²⁺ supplementation. In one embodiment, 2×10⁶ lymph node cells were mixed with BV421 conjugated-anti-mouse CD4, APC-Fire 750-conjugated anti-mouse CD8, APC-conjugated anti-mouse CD19 antibodies each at the predetermined dilution of 1:500, and the pH indicator pHrodo™ Green AM at the pre-determined dilution of 1:1000, and MitoSpy Orange at a final concentration of 100 nM in 100 ml LCIS supplemented with additional 0.7 mM CaCl₂ at 37 °C for 20 min. In another embodiment, Raji and Jurkat cells were stained for pHi, MMP and Annexin V in the same buffer at 37 °C for 15 min and chilled in ice water for 20 min.

### Control Immune Response According to its Natural Time Course

Mechanisms regulating an immune response, i.e., the control of death, proliferation, functions, migratory behaviors, and metabolism, etc. of the lymphocytes, may differ at different stages in the natural time course of an immune response. Therefore, to achieve effective treatment or prevention of immunological diseases one must target such mechanisms specifically or preferentially operating at the stage in the natural time course of the immune response at the time a subject seeks medical treatment. While not excluding the application of this strategy to other mechanisms of controlling an immune response, this invention focuses on how to control lymphocyte death and/or proliferation by modulating lymphocyte pHi by targeting the mechanisms for energy production operating in lymphocytes at the different stages in the natural time course of an immune response. Alternatively, it may be said that this invention provides methods and compositions to target energy metabolism in accordance with the pHi and MMP of the lymphocytes. In some embodiments, the treatments aim to lowering pHi to dampen overzealous immune response, which is considered to be the underlying cause of immunological diseases, including, but not limited to, allergic diseases, autoimmune diseases, and infectious diseases where immune response damages the structure and/or function of normal tissue or cells. In some embodiments, the treatment aims to raising pHi of lymphocytes to promote survival and/or proliferation of the lymphocytes thereby promote immune response to infection, cancer or vaccine.

In some embodiments, lymphocytes are targeted at the early stage in the natural time course to lower pHi to dampen overzealous immune response. At this stage, the lymphocytes are predominantly N lymphocytes that have high MMP and there is a strong inverse relation between MMP and pHi. Since the strong inverse relationship indicates that oxidization of carbons derived from pyruvates or fatty acids is the main contributor to the MMP, an effective strategy to lower pHi in lymphocytes in the early stage is to enhance influx of pyruvates and/or fatty acids to the TCA cycle so that the MMP is further increased, which in turn drives the pHi lower to induce apoptosis. Thus, in some embodiments, the increase of influx of pyruvates to the TCA cycle is induced by administering to a subject an inhibitor of pyruvate dehydrogenase kinase such as dichloroacetate (DCA) in a composition comprising 11.01 nM to 11.01M DCA in saline at a dose of 0.01 ml to 10 ml/kg bodyweight by i.p. injection or blood infusion. In some embodiments, the increase of influx of fatty acids to the TCA cycle is achieved by administering an activator of carnitine palmitoyltransferase-1 (CPT-1) such as C75 in a composition comprising 1.1 nM to 1.1M C75 in 5% DMSO and 95% saline at a dose of 0.01 ml to 100 ml/kg bodyweight.

Amino acid energy metabolism particularly glutaminolysis and glycolysis for energy metabolism (referred to as glycolysis for short) also contribute, likely more in the minority P lymphocytes than N lymphocytes, to energy production at this stage, but not to the extend to change the inverse relation between MMP and pHi even in the P lymphocytes. It was assumed that glycolysis by itself would not increase protons in the cells, but it would decrease ATP production from mitochondria, therefore indirectly counter the downward pressure on pHi in lymphocytes at the early stage. In contrast, it was assumed that deamination of amino acids, export of the deaminated carboxylates, and participation in the malate-aspartate shuttle by the downstream metabolites of the amino acids can remove protons therefore increase pHi. Accordingly, inhibiting these pathways can help lower pHi. Therefore, in some embodiments inhibitors of amino acid energy metabolism and/or glycolysis are used alone or in combination with regulator(s) of energy metabolism that increase the influx of pyruvates and/or fatty acids to the TCA cycle to lower pHi. In some embodiments, an inhibitor of glutaminase such as CB-839 is administered into a subject by i.p. injection or blood infusion in a composition comprising 11 nm to 11M CB-839 in 5% DMSO and 95% saline at a dose of 0.01 ml to 100 ml/kg bodyweight to inhibit glutaminolysis. In some embodiments, an inhibitor of lactate dehydrogenase such as GSK2837808A (GSK in short) is administered to a subject by i.p. injection or blood infusion in a composition comprising 52.8 μM to 52.8 mM GSK in 5% DMSO and 95% saline at a dose of 0.01 ml to 100 ml/kg bodyweight to inhibit glycolysis. In some embodiments, the glutaminolysis inhibitor(s) or other inhibitor(s) of amino acid energy metabolism and glycolysis inhibitor(s) may be administered together to a subject. In some embodiments, the inhibitor(s) of amino acid energy metabolism and/or glycolysis may be administered together with promoter(s) of the influx of pyruvates and/or fatty acids to the TCA cycle.

In some embodiments, treatments are aimed to raise pHi of lymphocytes at the early stage of the natural time course of immune response to promote survival and proliferation of lymphocytes to combat infection or cancer. In some embodiments, the pHi is raised by promoting amino acid energy metabolism such as glutaminolysis and/or glycolysis. In some embodiments, influx of pyruvates and/or fatty acids to TCA cycle is restricted. In some embodiments, the combination of these two strategies are employed.

The intermediate stage of the natural time course of immune response is marked by the transition to P lymphocytes with a positive relation between MMP and pHi. At such transitional state, lymphocytes are expected to be highly amenable to metabolic manipulation. Importantly, the positive relation between MMP and pHi indicates that catabolism of the backbones of amino acids, for example, alpha-ketoglutarate derived from glutamine, in the TCA cycle has become a major contributor to the MMP of the lymphocytes. Because it is assumed that the overall reactions of breaking down amino acids for energy production reduce protons in the cells, higher MMP no longer equal to more proton accumulation. Therefore, in some embodiments, dampening overzealous immune response is highly effectively achieved by attenuating the transition to P lymphocytes with a positive relation between MMP and pHi with inhibitor(s) of amino acid energy metabolism. In some embodiments, the inhibitor of amino acid energy metabolism is the inhibitor of glutaminase CB-838. CB-839 is administered into a subject by i.p. injection or blood infusion in a composition comprising 11 nm to 11M CB-839 in 5% DMSO and 95% saline at a dose of 0.01 ml to 100 ml/kg bodyweight to inhibit glutaminolysis. In some embodiments, entry of carbon backbones of amino acids may be restricted or blocked. To this end, in some embodiments, the alpha-ketoglutarate dehydrogenase inhibitor CIP-613 is administered to a subject in a composition comprising 0.11 nM to 0.11M CIP-613 in 5% DMSO and 95% saline at a dose of 0.01 ml to 100 ml/kg bodyweight. Glycolysis is an additional way of ATP production at the intermediate stage. Therefore, in some embodiments, inhibitor(s) of glycolysis such as GSK is administered to a subject by i.p. injection or blood infusion in a composition comprising 52.8 μM to 52.8 mM GSK in 5% DMSO and 95% saline at a dose of 0.01 ml to 100 ml/kg bodyweight to lower pHi.

Furthermore, influx of pyruvates and fatty acids to the TCA cycle is expected to continue to contribute substantially to MMP. Therefore, in some embodiments, dampening overzealous immune response at the intermediate stage is also effectively achieved with promoter(s) of influx of pyruvates and/or fatty acids to TCA cycle. However, unlike the inhibition of amino acid energy metabolism, increase of influx of pyruvates or fatty acids to TCA does not attenuate the transition to P lymphocytes with a positive relation between MMP and pHi. In some embodiments, the promoter of the influx of pyruvates to TCA cycle is DCA. DCA is administered to a subject in a composition comprising 11.01 nM to 11.01M DCA in saline at a dose of 0.01 ml to 10 ml/kg bodyweight by i.p. injection or blood infusion. In some embodiments, the promoter of the influx of fatty acids to TCA cycle is C75. C75 is administered to a subject by i.p. injection or blood infusion in a composition comprising 1.1 nM to 1.1M C75 in 5% DMSO and 95% saline at a dose of 0.01 ml to 100 ml/kg bodyweight.

In some embodiments, various combinations of the above-described compounds may be administered to a subject to dampen overzealous immune response. In some embodiments, amino acid energy metabolism such as glutaminolysis is stimulated to facilitate the transition to P lymphocytes with a positive relation between MMP and pHi and/or to raise pHi to reduce apoptosis or promote survival of the lymphocytes, therefore, enhance immune response to infection or cancer.

At the late stage in the natural time course of immune response, most of the lymphocytes have transitioned to P lymphocytes with a positive relation between MMP and pHi. Thus the P lymphocytes will be the primary target for manipulating immune response at this stage. As described above, it is assumed that the positive relation between MMP and pHi indicates that oxidation of carbon backbones of amino acids is a major contributor to MMP or ATP production in the P lymphocytes. In addition oxidation of carbons derived from the fatty acid, or in other words influx of fatty acids to TCA cycle, is also a major contributor to MMP and ATP production in the P lymphocytes at the late stage.

Thus, at the late stage, in some embodiments, dampening overzealous immune response is accomplished by administering to a subject inhibitor(s) of amino acid energy metabolism and/or glycolysis such as CB-839 and/or GSK in compositions and at doses described above. In some embodiments, dampening immune response is accomplished with inhibitor(s) of entry of carbon backbones of amino acids and/or the influx of fatty acids to TCA cycle. While not wish to be bound to any particular theory, data disclosed herein suggest such treatment(s) induce imbalance between ATP synthesis and hydrolysis. In some embodiments, the inhibitor of entry of carbon backbones of amino acids to TCA cycle is CIP-613. It is administered to a subject in the composition and dose described above. In some embodiments, the inhibitor of the influx of fatty acids to the TCA cycle is Etomoxir (Etom). It is administered to a subject in a composition comprising 0.55 μM to 0.55M Etom in 10% DMSO and 90% saline at a dose of 0.01 to 100 ml/kg bodyweight. In some embodiments, particularly but not exclusively in cases where substantial portion of the minority N lymphocytes have high MMP and strong inverse relation between MMP and pHi such as in the late-e2 or e3, promoter(s) of the influx of pyruvates and/or fatty acids such as DCA and C75, respectively, are administered to a subject to dampen overzealous immune response. DCA and/or C75 are administered in compositions and doses described above. In some embodiments, various combinations of the regulators of energy metabolism described in this paragraph are administered to a subject to dampen overzealous immune response. Further, the presence of the small n lymphocytes at the late stage is an indication of insufficient ATP synthesis from mitochondrial respiration to power high rates of proliferation in these cells. In the late-e1 and e2 states, the n lymphocytes remain highly responsive to treatment with promoter(s) of influx pyruvates and/or fatty acid to the TCA cycle so that their MMPs, as well as those of other lymphocytes, are elevated by such treatments. Therefore, in some embodiments, in cases, albeit not exclusively, where the n lymphocytes are detected, treatment(s) with inhibitor(s) of amino acid energy metabolism and/or glycolysis, and/or treatments with inhibitor(s) of the entry of carbon backbones of amino acids and/or influx of fatty acids to the TCA cycle are combined with promoter(s) of the influx of pyruvates and/or fatty acids to the TCA cycle to dampen overzealous immune response.

### Combinatorial Approach to Lowering pHi for Cancer Therapy

Metabolic plasticity provides cancer cells an advantage for survival and growth. It also presents a great challenge for cancer therapeutics that target a single metabolic pathway. Cancer cells can adopt a different metabolic pathway to make such treatment ineffective. Even without this consideration, therapeutics targeting a single pathway would require near complete blockade of the pathway to achieve high therapeutic efficacy. This would require very high dose of inhibitor because cancer cells often have elevated expression of the enzymes required for their favored energy production pathway. Since most metabolic pathways are shared by cancer and normal cells, near complete blockade of a pathway with a high dose of inhibitor is likely to cause severe adverse side effect. Data disclosed in this application have shown that there are multiple ways to lower pHi to induce apoptosis. This offers an opportunity to lower pHi of cancer cells by partially modulating multiple pathways. While the partial modulation of any single pathway may not be enough to induce apoptosis, partial modulation of multiple pathways could collectively lower pHi of the cancer cells enough to induce apoptosis. Since no single pathway is blocked to near completion or driven over the edge, this strategy is less likely to cause adverse side effects while in the meantime achieves high therapeutic efficacy.

Based on this hypothesis, this invention claims that a panel of compositions of the mixtures of low amounts of multiple components that target different metabolic pathways (referred to as combinatorial compositions) together with or without the individual components of the mixtures be created. This panel of compositions is screened for the inhibition of the growth (increase of cell number) and/or survival of the cells of the cancer to be treated. This screening may or may not be coupled with a screening for the inhibition of the growth and/or survival of normal cells. The composition(s) that show strong inhibition of the cancer cells with or without also showing relatively weak or no inhibition (as compared with the average of all compositions and vehicle-treated controls) of normal cells are selected and provided in required quantities for treating the tested cancer in the subject. In some embodiments, the normal cells used in this screening procedure are peripheral leukocytes, for example those derived from the subject's peripheral blood or healthy donor(s), that are stimulated with concanavalin (ConA) or phytohemagglutinin (PHA) and/or lipopolysaccharide (LPS) together with or without interleukin-2 and/or interleukin-4. In some embodiments, the normal cells are a mixture of allogenic leukocytes. In some embodiments, the pathways targeted by the panel of compositions are the influx of pyruvates to the TCA cycle, the influx of fatty acids to the TCA cycle, the entry of carbon backbones of amino acids to the TCA cycle, amino acid energy metabolism, glycolysis for energy production. For the first time, data in this invention have demonstrated that manipulating these pathways alone or in combination can lower pHi of the cancer cells to induce apoptosis. In some embodiments, the compounds that target the aforementioned pathways and used to make the combinatorial compositions are inhibitors of lactate dehydrogenase, inhibitors of malate-aspartate shuttle, inhibitors of glutaminase, inhibitors of pyruvate dehydrogenase kinase, inhibitors of alpha-ketoglutarate dehydrogenase, inhibitors of carnitine palmitoyltransferase, activator of carnitine palmitotransferase. In some embodiments, the compositions for cancer therapy are compositions 8′, 12′, 15, 16′, 19 and 21′ listed in Table 1, their individual components and exemplary concentrations, which may be adjusted up or down, are provided in Table 1.

For the convenience in clinical application, individual compounds and mixtures of their various combinations, along with other necessary agents such as those for measuring cell viability, growth/proliferation, pHi, and MMP, and instructions for the assays may be provided to health care professionals as a kit, together with or without related equipment and laboratory tools.

Ki-67 as a Predictive Marker for Responsiveness to Low pHi-Based Cancer Therapy (Switch to Feb. 12 2024 Corrections Accepted Version for Translation)

Biomarker with predictive value for responsiveness to therapeutics remains elusive for most cancers. Ki-67 is a reliable marker for proliferating cells whose level of expression positively correlates with rRNA and DNA synthesis (R43, 24), but is not a clear predictor of responsiveness to existing chemotherapeutics. However, in this invention Ki-67 will be used as predictive biomarker for responsiveness of a subject to the new low pHi-based cancer therapy. Low pHi-based cancer therapy refers to cancer therapy by lowering the pHi of the cancer cells to induce cancer cell death. High level of Ki-67 and/or high percentage of Ki-67+ cancer cells predicts positive response to the low pHi-based cancer therapy. In some embodiments, the expression of Ki-67 is detected and measured by flow cytometry of cancer cells that have been intracellularly stained for Ki-67. The cancer cells may also be stained for marker(s) that help identify the cancer cells from normal cells. In some embodiments, Ki-67 is detected by immunohistochemistry staining of tumor tissues together with staining of marker(s) that identify the tumor cells. In some embodiments, Ki-67 transcripts are measured using techniques such as real-time RT-PCR. For convenience of clinical application, all reagents and instructions for the assays may be provided to the heath professionals as a kit.

Treat Skin and Mucosa Infections with pH Modifier(s)

Some skin and mucosa infections such as fungal infections of the nails are very difficult to treat. This invention offers a solution to this problem. In some embodiments, microbial infections of the skin (including skin appendages such as nails) and mucosa are treated by topical application of a composition comprising pH modifier(s) that lower pH. Skin and mucosa infections include but are not limited to onychomycosis, tinea pedis, impetigo, cellulitis, gonorrhea, etc. In some embodiments, onychomycosis is treated by topical application of a composition of pH modifier(s) that lower pH to the infected and surrounding areas. In some embodiments, the pH modifier that lowers pH is HOAc, and it is used in a composition comprising 0.01 to 100% HOAc by volume in water. In some embodiments, tinea pedis is treated by topical application of a composition comprising 0.01% to 100% HOAc by volume in water, and in some embodiments the infection is treated by submerging the infected area(s) in the composition diluted in water. In some embodiments, mucosa infection by known or unknown pathogens is treated by topical application of a composition comprising 0.01 to 100% HOAc by volume in water. The compositions described above may be diluted to a given subject's preference.

Treat Neoplasia of the Skin with pH Modifier(s)

Neoplasia of the skin such basal cell carcinoma is usually treated by surgery and radiation therapy. These treatments are not only stressful, in cases where the neoplasia is on the skin of the face patients may also be reluctant to accept certain therapies such as surgery. Here, this invention provides an alternative that is essentially stress-free and has little effects on a subject's appearance. Thus, in some embodiments, skin neoplasia such as basal cell carcinoma, is treated by topical application of compositions composing pH modifier(s) that lower pH in an aqueous environment. In some embodiments, the pH modifier is HOAc in a composition comprising 0.01% to 100% HOAc by volume in water. The compositions described above may be diluted to a given subject's preference. In some embodiments, pH modifier(s) may be injected subcutaneously to facilitate the delivery of the pH modifiers to the neoplastic cells. In some embodiments, superficial incision(s) are introduced to the lesion prior to topical application also to facilitate the delivery of the pH modifier(s) to the neoplastic cells.

### EXAMPLES

### Materials and Methods

### Mice and Animal Models of Diseases

Balb/c and C57BL/6 mice were purchased from Jackson Laboratory (Bar Harbor, ME, USA), housed and bred at the animal facility of Therazwimm Corporation. Animal studies were performed according to the protocols approved by Institutional Animal Care and Use Committee of Therazwimm. Asthma induction and analyses were performed essentially as previously described. (26). For sensitization, grade II ovalbumin (OVA) (Sigma-Aldrich, St. Louis, MO, USA) and Alum adjuvant (Thermo Scientific, Rockford, IL, USA) were fresh mixed in PBS to make sensitization solution comprising 200 μg/ml OVA and 33.4% (by volume) Alum. Adult mice were i.p. (intraperitoneally) injected with 100 μl of the sensitization solution (20 μg OVA/mouse) on day 0. On day 13, each mouse was sensitized again by i.p. injection of 500 μl of fresh prepared sensitization solution (100 μg OVA/mouse). Unless specified otherwise, on days 13 and 14 after the second sensitization, mice were challenged with OVA (100 μg/mouse) in 80 μl saline by intra tracheal injection. Mediastinal and parathymic lymph nodes (collectively referred to as mediastinal lymph nodes or MLNs in this application) that drain the lungs, (27), were collected as draining lymph nodes (DLNs), cervical, facial and/or inguinal lymph nodes were collected as non-draining lymph nodes (NDLNs).

Induction of experimental autoimmune encephalomyelitis (EAE) in C57BL/6 mice was carried out as previously described. (28). Antigen emulsion was prepared by mixing myelin oligodendrocyte glycoprotein peptide MOG₃₅₋₅₅ (referred to as MOG for short) (G. L. Biochem, Shanghai, China), desiccated M. tuberculosis H37Ra (Difco Laboratories, Detroit, MI, USA) and incomplete Freund's adjuvant 11 (IFA) (Difco Laboratories) in PBS. The final antigen emulsion comprised 1.5 mg/ml of MOG, 189 μg/ml of M. tuberculosis H37Ra and 50% (by volume) IFA. On day 0, mice were immunized by s.c. (subcutaneous) injection of the antigen emulsion at 1 site each in front and back the dorsal areas (100 μl/site). Two hours later, the mice were i.p. injected with Pertussis Toxin (List Biological laboratories, Campbell, CA, USA) freshly prepared in ice-cold PBS (200 ng in 100 μl PBS/mouse). On day 1, the mice received a second i.p. injection of Pertussis Toxin at the same dosage. Starting on day 10, at which time no disease onset was observed, mice were scored for clinical symptoms as described. (28). Mice with clinical score of 3 or above were provided wet chows.

### Airway Hyperresponsiveness (AHR) Assay

AHR was measured as previous described (26). Briefly, 4 days after the last OVA challenge, individual mice were anesthetized, and a small cannula was inserted into the trachea. The mice were mechanically ventilated with the Buxco Elan Series RC instrument (200 breath per min; 0.2 ml tidal volume), and challenged with inhalation of increasing concentrations of aerosolized solutions (10 μl) of methacholine (6.25, 12.5, 25, 50 mg/ml, 3 minutes for each challenge).

Treatment of Mice with Compositions of Metabolic Regulator(s)

Mice were i.p. injected with 200 μl of the composition listed in Table 1 at the time indicated in each experiment. For this purpose, the day when the mice received the first challenge in the round where treatments were applied or tissues were harvested was designated as day 0. Mice were typically 29 sacrificed 20 to 24 hours after the last injection.

### Culture of Tumor Cells

Jurkat and Raji tumor cell lines were maintained in Complete RPMI-1640 Medium (RPMI-1640 plus 1×GlutaMAX, 100U/ml Pen-Strep (Gibco Life Technologies, Grand Island, NY, USA), and 5% heat-inactivated fetal bovine serum (FBS) (Atlanta Biologicals, Flowery Branch, GA, USA) at 37 °C and 5% CO2. For analyzing the effect of HOAc treatment on pHi, the tumor cells (2×10⁶ cells/ml) were incubated in FBS comprising 10% saline alone or 87.5 mM HOAc or NaOH in slaine in a 37 °C water bath for 20 minutes. For studies of the inhibition of tumor cell growth, cells in exponential growth phase were collected and seed at 2.5×10⁵ cells/ml in the Complete RPMI-1640 medium, composition of metabolic regulator(s) listed in Table 1 was added at 1:10 dilution. After culture for 2 days, half of medium was replaced with fresh medium with the same concentrations of the metabolic regulator(s). One day later, the cells were analyzed by flow cytometry for live cell count, pHi, MMP and apoptosis.

### In Vitro Culture of Lymphocytes and Treatments

Lymph node cells of unimmunized mice were cultured in Complete RPMI-1640 medium at 2×10⁶ cells/ml in flat bottom 96-well plate with ConA (2.50 g/ml), LPS (50 g/ml), IL-2 20 units/ml and IL-4 (10 ng/ml). Compositions listed in Table 1 were added to the culture at 1:10 dilution. On day 3 post stimulation, cells were harvested and analyzed by flow cytometry for live cell count, pHi, MMP and apoptosis.

### Flow Cytometry

Fluorescence-conjugated antibodies against mouse CD4, CD8, CD19, Ki-67; Zombie-Green fixable viability kit, Zombie-Violet fixable viability kit; MitoSpy Orange, fluorescence- or biotin-conjugated Annexin V, 7AAD, fluorescence-conjugated Streptavidin, and Foxp3 staining buffer set were purchased from Biolegend (San Diego, CA, USA). PE-conjugated anti-human Ki-67 antibody was purchased from eBioscience (San Diego, CA, USA). pHrodo™ Green AM and pHrodo™ Red AM and the PowerLoad was purchased from ThermoFisher Scientific (Waltham, MA).

For live cell count, lymph nodes or tumor cells were prepared as single cell suspension in the same volume of buffer. An aliquot of the same volume of cells of different samples were stained with 7AAD with or without other staining. The cells were analyzed by flow cytometry with the same acquisition volume, flow speed and acquisition time.

For staining lymphocytes for pHi, MMP, Annexin V and surface markers CD4, CD8 and CD19, the cells were washed once with serum-free Annexin V binding buffer, then incubated in 100 l Annexin V binding buffer or Live Cell Image Solution (LCIS) (ThermoFisher Scientific, Waltham, MA) supplemented with 0.7 mM CaCl₂) containing fluorescence-conjugated antibodies, Annexin V, pHrodo™ Green and MitoSpy Orange at predetermined dilutions in a 37 °C water bath for 20 min. After washing with ice-cold Annexin V binding buffer plus 1% FBS, cells were resuspended in 100 l Annexin V binding buffer or the Ca²⁺-supplemented LCIS. 7AAD was added prior to flow cytometry. For Ki-67 staining, sorted live lymphocytes were fixed and permeabilized using Foxp3 staining buffer kit, then stained with fluorescence-conjugated anti-mouse Ki-67 antibodies. (Biolegend, San Diego, CA, USA).

Flow cytometry data acquisition was carried out using Attune™ cytometer (Invitrogen, Carlsbad, CA). Data analyses were performed using FlowJo X.

### Intracullar ATP Counts

Lymphocytes were washed twice with ice-cold plain PBS, and freeze-thawed twice in 150 μl PBS to release intracellular ATP. The lysates were briefly centrifuged at 10,000 rpm in a microfuge. Supernatants were recovered and 50 μl of which was used for luciferase assay with the RealTime-Glo™ extracellular ATP assay reagent (Promega, Madison, WI, U.S.A.).

### Example A. Determine the Natural Time Course of Immune Response

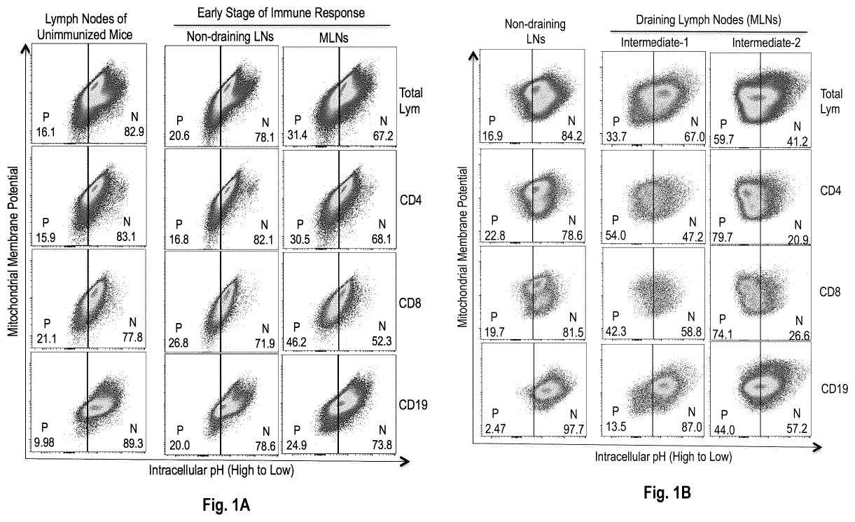

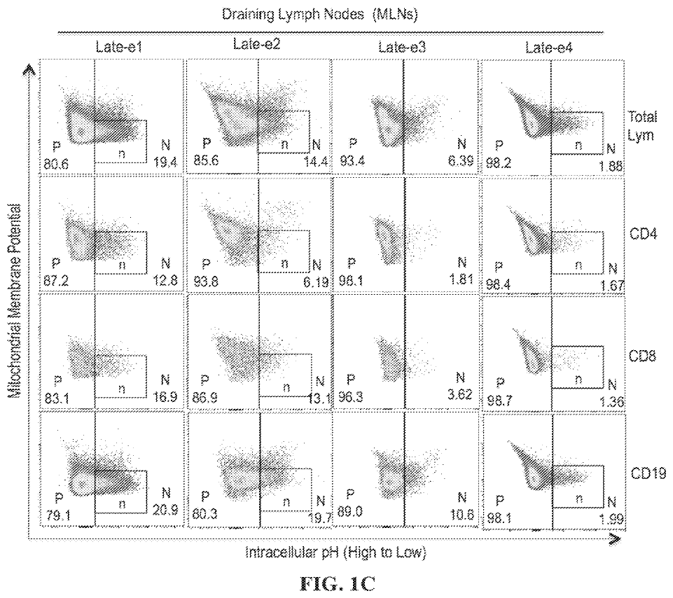

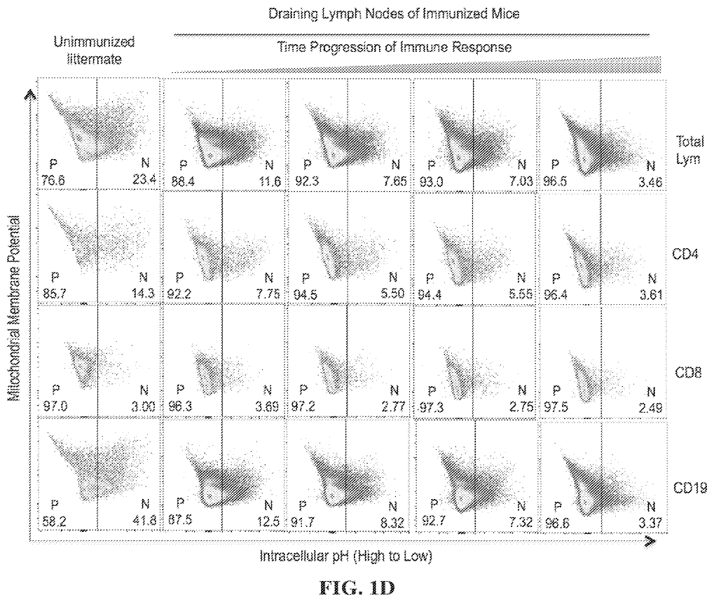

In the mouse model of asthma, pH and MMP of lymphocytes in the non-draining lymph nodes (NDLNs) and the mediastinal lymph nodes (MLNs) that drain the lungs were analyzed in a timely progressive manner following intra-tracheal challenges with OVA. The lymphocytes were divided into P and N populations that had high and low pHi, respectively. (FIGS. 1A-1C). At the early stage, all lymphocytes regardless of in the P or N populations showed an inverse relation between MMP and pHi that is in turn inversely related to the fluorescence intensity of the pHi indicator pHrodo™ Green. Nonetheless, compared with unimmunized mice (no OVA sensitization and challenge), the percentages of N populations were lower. Within the same immunized mice, the N populations in the MLNs were further reduced as compared with those in the NDLNs. Nonetheless the N populations are the dominant lymphocyte populations. Thus, the early stage is defined by the dominance of the N population and the inverse relation between MMP and pHi in all lymphocytes. (FIG. 1A).

As time progressed, the immune response in the MLNs entered the intermediate stage. (FIG. 1B). At this stage, lymphocytes in the P population displayed a positive relation between MMP and pHi. However the N population may or may not still remain as the dominant lymphocyte population, and substantial portion of them had high MMP. It must also be noted that not all lymphocyte subpopulations entered the intermediate stage at the same time. In the asthma model, at the time points of analyses, the CD4 and CD8 T cells in MLNs had entered the intermediate stage, whereas the B cells remained at the early stage. (FIG. 1B). In the intermediate-2 stage, the T cells in the P populations had differentiated into 2 distinct populations with relatively higher and lower MMP and pHi, respectively. (FIG. 1B).

Following the intermediate stage, the percentages of N populations further declined and the P populations became overwhelmingly the dominant populations. At the late stage, the N populations may continue to decline. In the asthma model, late stage lymphocytes were detected in one of four distinct energetic states, named as late-e1, e2, e3 and e4. (FIG. 1C). The late-e1 state is characterized by substantial numbers of N lymphocytes that have low MMP and show weak or no correlation between MMP and pHi, these cells are termed the “n” cells. Nonetheless there are some N lymphocytes with intermediate levels of MMP and an inverse relation between MMP and pHi. In the late-e1 state, the P lymphocytes showed low to intermediate levels of MMP. (FIG. 1C). In the late-e2 state, the n cells can still be detected within the N populations, but they are not as prominent as in the late-e1 state. Importantly, the MMPs in both the P and N lymphocytes were elevated as compared with those in the late-e1 state. (FIG. 1C). In contrast, the n cells were essentially absent in the late-e3 state, and the P and N lymphocytes show steep (strong) positive and negative correlation between MMP and pHi, respectively. (FIG. 1C). Both MMP-high and low P lymphocytes are substantially detected in late-e2 and e3. (FIG. 1C). In late-e4 state, almost all N lymphocytes had turned into n cells, few, if any, showed strong correlation between MMP and pHi. Similarly, most P lymphocytes had levels of MMP comparable to that of the n cells instead of segregating into distinct populations. (FIG. 1C).

However, the characterization of the immune response based on the profiles of pHi and MMP of the lymphocytes should not be construed as that every immune response must go through all the different stages. On the contrary, depending on the history and degree of exposure to environment antigens, an immune response may start at a stage after the afore-described early stage. For example, in a study with the EAE model, lymphocytes of unimmunized littermates showed profiles of MMP and pHi similar to those of the late-e2 state described in the asthma model. (FIG. 1D). This indicates that the mice had active ongoing immune response prior to immunization with specific antigen. Nonetheless, after immunization, the N population of the lymphocytes in the draining lymph nodes (DLNs) progressively declined even though they had skipped the early and intermediate stages. (FIG. 1D).

### Example B. Control Immune Response at the Intermediate Stage in Asthma Model

Although the dynamic changes of pHi and MMP in lymphocytes reflect the natural time course of immune response, for practical purpose, one may not have to rely on the concept of natural time course of immune response, but rather relies on the profiles of pHi and MMP of the lymphocytes to design therapeutic strategy. Such different narratives should not be considered to be materially different from the current disclosure that in essence stresses the use of the profiles of pHi and MMP of the lymphocytes to guide the treatments of immunological diseases.

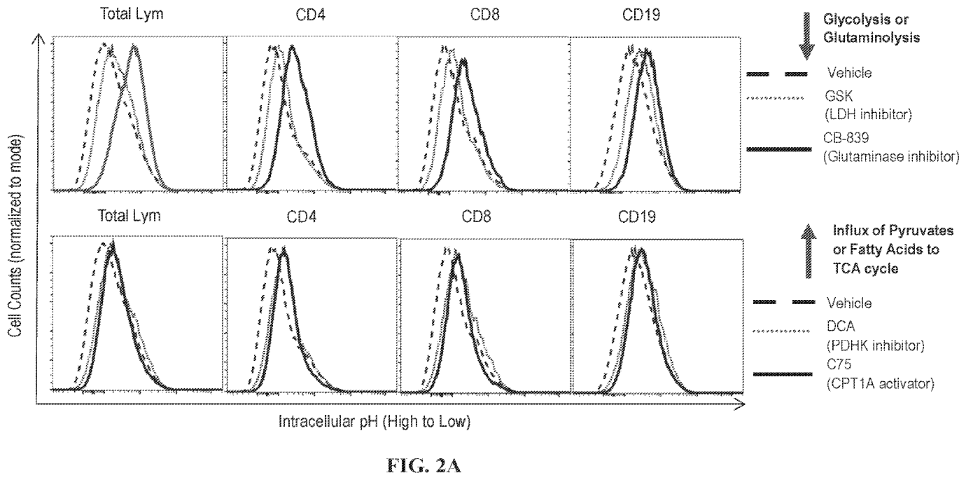

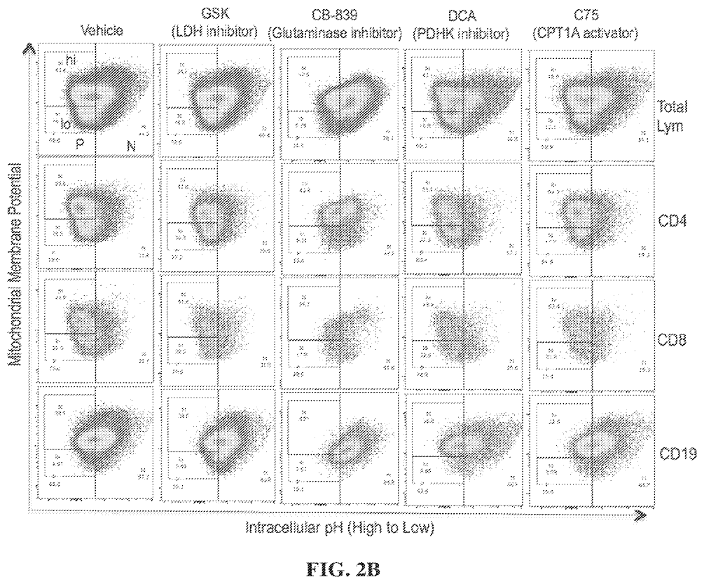

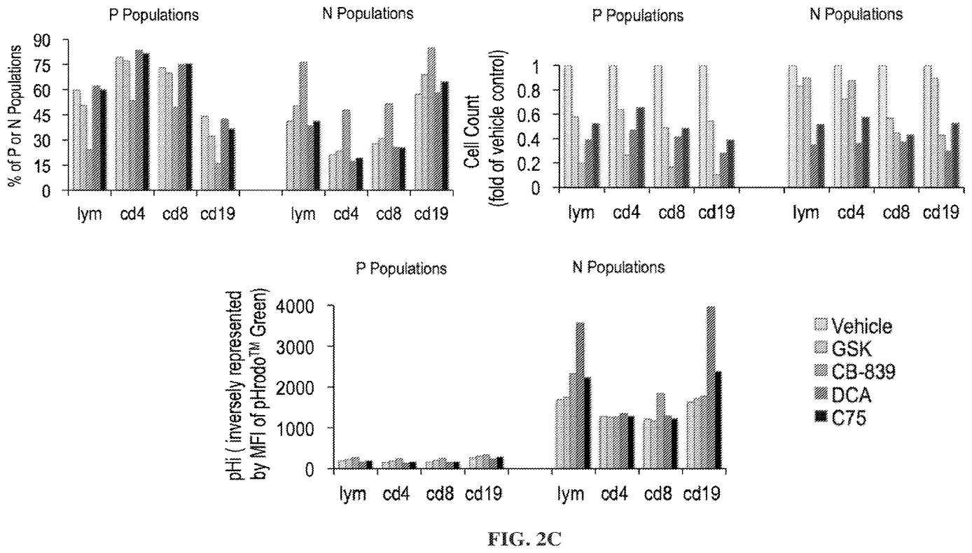

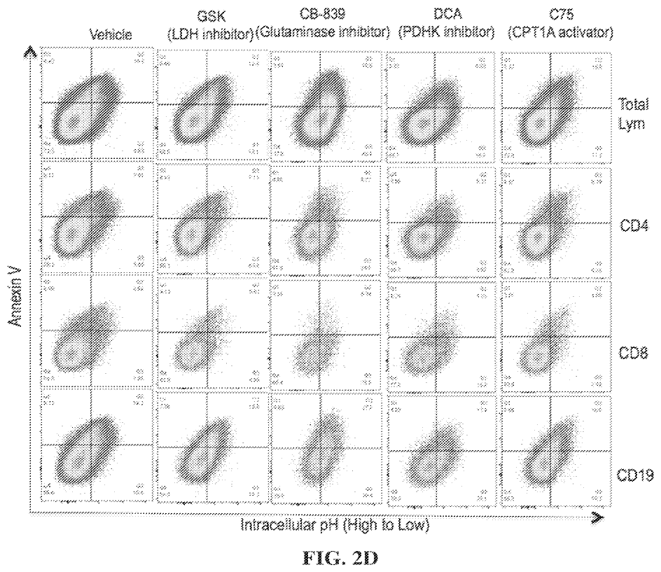

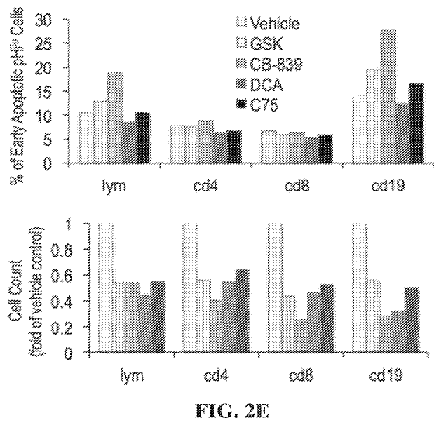

At the intermediate stage lymphocytes are transitioning to P lymphocytes with a positive correlation between MMP and pHi. Two strategies were designed to lower the pHi of the lymphocytes, which would in turn induce apoptosis of the lymphocytes therefore dampen immune response to the experimental allergen OVA. One strategy was to inhibit glycolysis for energy production or amino acid energy metabolism such as glutaminolysis. Glycolysis was inhibited by i.p. injection of the lactate dehydrogenase inhibitor GSK-2837808A (GSK for short), whereas glutaminolysis was inhibited by i.p. injection of the glutaminase inhibitor CB-839. The other strategy was to enhance the influx of pyruvates and/or fatty acids to the TCA cycle. The influx of pyruvates was enhanced by i.p. injection of the pyruvate dehydrogenase kinase inhibitor dichloroacetate (DCA), and influx of fatty acids was enhanced by i.p. injection of the carnitine palmitoyltransferase-1 (CPT-1) activator C75. Neither of these two strategies had been shown before to lower pHi to induce apoptosis of lymphocytes. As shown in FIG. 2A, both strategies were effective in lowering the pHi of the lymphocytes in the MLNs of mice sensitized and challenged with OVA. One may speculate that these compounds modulated mitochondrial respiration, but it should be noted that not any compound that modulates mitochondrial respiration could have the same or similar effects (data not shown).

The effects of the compounds on the magnitude of immune response were assessed by directly counting the numbers of live lymphocytes in the MLNs and measuring the percentages of early apoptotic cells. The latter was used as proxy of apoptosis because pHi could not be measured in apoptotic (dead) cells due to their leaky cell membrane. However, the percentages of early apoptotic cells could underestimate the effects of the compounds because dead cells are quickly removed in vivo, (29), and may disintegrate during in vitro experiments therefore are often unaccounted for in such assays. Therefore, live cell count is critically important and must be considered when interpreting data of early apoptosis.

The effects of the compounds on the P and N populations are shown in FIG. 2B and the numerical presentation of the same data in FIG. 2C. The percentages and the absolute numbers of live P lymphocytes were greatly diminished by the inhibition of glutaminolysis and to lesser degree by inhibiting glycolysis at their respective doses used in this study. (FIG. 2C, upper and middle panels). Therefore, the transition to P lymphocytes and their acquisition of the positive correlation between MMP and pHi is heavily dependent on glutaminolysis. In the N populations, inhibition of glycolysis or glutaminolysis had only minor effects on CD4 T cells, but inhibition of glutaminolysis greatly reduced the absolute number of live CD8 T and B (CD19+) cells. Treatment with DCA or C75 both significantly reduced both the P and N lymphocytes. (FIG. 2C, middle panel). Early apoptotic cells are shown in FIG. 2D and numerically presented in FIG. 2E. It is noteworthy that the early apoptotic cells were enriched in the pHi-low cells. (FIG. 2D). While all compounds greatly reduced the live lymphocytes, increase of early apoptotic cells was detected in some treatment groups, (FIG. 2E), indicating that this assay under estimated the effects of the compounds. Enhancing influx of pyruvates to the TCA cycle greatly reduced the pHi of the B cells in the N population, which had higher MMP than the T cells even in the vehicle controls. This may explain why enhancing pyruvate influx caused the greatest reduction of B cells in the N population than any of the other treatments. (FIG. 2C).

### Example C. Control Immune Response at the Early Stage in Asthma Model

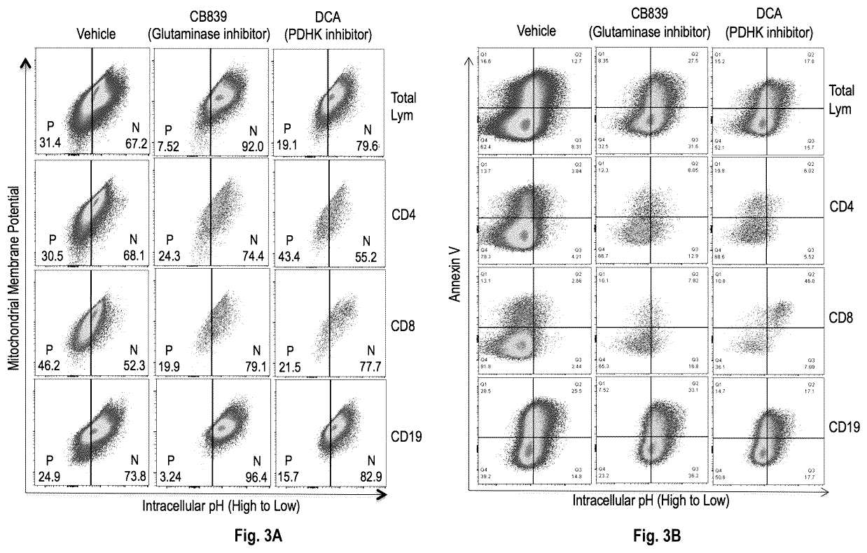

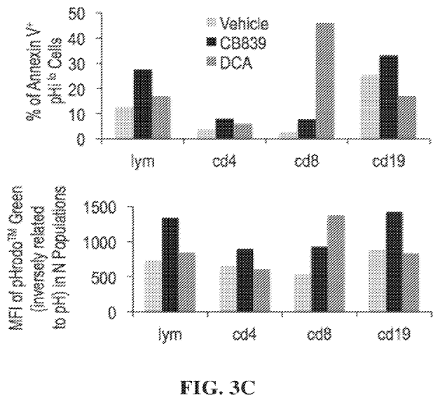

Example B showed that the glutaminolysis inhibitor CB-839 and the pyruvate influx enhancer DCA at the doses tested were the most effective in down regulating the immune response to OVA challenges. In this example, these two compounds were also tested at the early stage of the immune response. Both compounds caused dramatic shifts of the pHi to the lower side so that the percentages of N populations were increased in live total lymphocytes and the T and B cell subpopulations. (FIG. 3A). Early apoptosis was again correlated with low pHi (FIG. 3B). CB-839 increased the percentages of early apoptotic cells in all lymphocyte populations, whereas DCA increased early apoptosis in T but not in the B cells. The changes in early apoptosis correlated with the changes of the pHi values in the N lymphocytes. (FIG. 3C).

### Example D. Control Lymphocytes Populations in the Late-e2 State in Asthma Model

First set of studies were focused on the late-e2 state because in this state there are various subpopulations of lymphocytes, including those with high MMP, negative or positive correlation between MMP and pHi, and n lymphoyctes. (FIG. 1C). In addition to DCA and C75, CPI-613 and Etomoxir (Etom for short) were also studied. CPI-613 is an inhibitor of alpha-ketoglutamate dehydrogenase (α-KGDH) and pyruvate dehydrogenase. However since pyruvate can enter the TCA cycle through pyruvate carboxylase alternative pathway, CPI-613 was expect to mainly block the entry to the TCA cycle of the carbon backbones of amino acids such as glutamine that use alpha-ketoglutarate as the entry point to the TCA cycle. Etom is an inhibitor of CPT-1, thus an inhibitor of fatty acid influx to the TCA cycle.

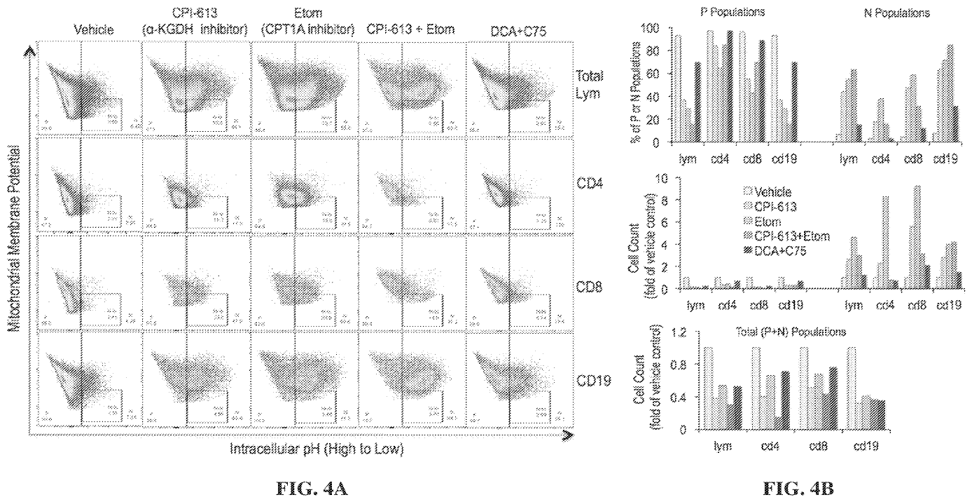

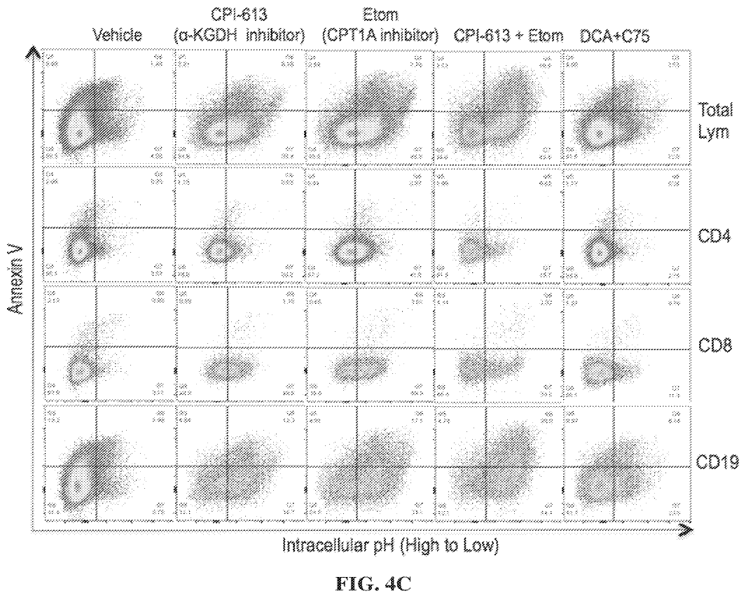

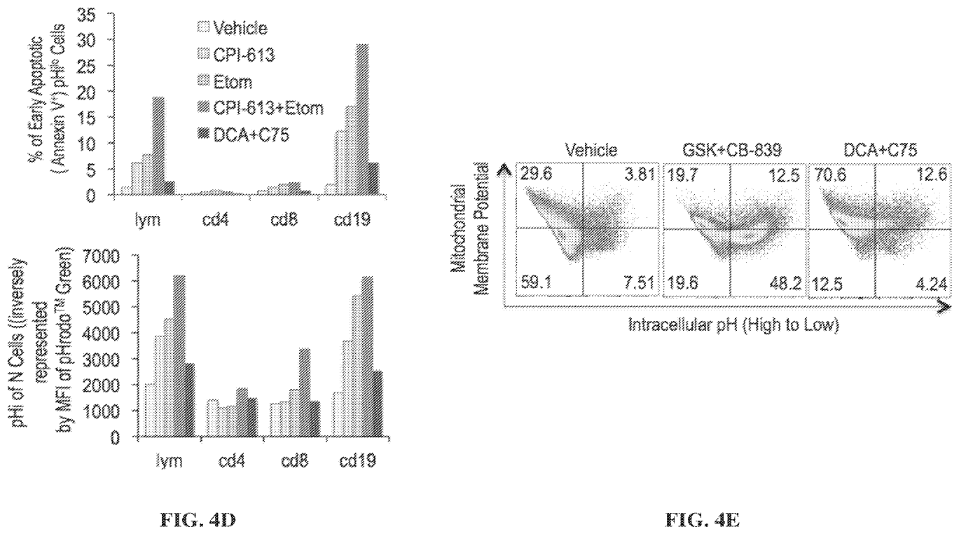

The effects of CPI-613 and Etom individually and in combination, and the combination of DCA and C75, on the distribution of the P and N populations are shown in FIG. 4A, and the percentages of the P and N populations are shown in FIG. 4B. CPI-613 and Etom individually or in combination decreased the percentages and absolute numbers of all lymphocytes in the P populations. (FIG. 4B, upper and middle panels). This result demonstrated a non-redundant role of energy production from amino acid backbones and fatty acids in the P lymphocytes. Conversely, these two compounds individually and in combination increased the N populations both in percentages and in absolute numbers. (FIG. 4B, upper and middle panels). Therefore, reduction of carbon oxidation of amino acid and/or fatty acids in the TCA cycle might have converted the P lymphocytes to N lymphocytes, and the P and N lymphoyctes responded in mutually opposite manners to such treatments.

In contrast, the combination of DCA and C75 showed only mild reduction of the P lymphocytes, and slight increase of the CD8 T cells and B cells in the N populations. When total live lymphocytes, T and B cells were analyzed, strongest reduction of the numbers of CD4 and CD8 T cells was achieved with the combination of CPI-613 and Etom, whereas reduction of live B cells was comparable among all treatments. Substantial reduction of the absolute numbers of live T cells and total lymphocytes was also observed in all other treatments. (FIG. 4B, lower panel). Since the combination of DCA and C75 increased the absolute numbers of the N lymphocytes, the reduction of the total live lymphocytes populations by this combination was likely the result of reduction the P lymphocytes.

Further analyses of early apoptosis showed again the correlation between early apoptosis and low pHi. (FIG. 4C). The treatments increased the percentages of early apoptotic cells, and the degrees of increase were consistent with the degrees of decreases of pHi in the N populations. (FIG. 4D). The effect of the combination of GSK+CB-839 that inhibits glycolysis and glutaminolysis on the lymphocyte populations in the late-e2 state was also investigated. This treatment decrease the percentage of P lymphocytes, with much more reduction of the P lymphocytes with low MMP than those with high MMP, whereas it increased the percentage of the N lymphocytes with more increase of those of low MMP than those with high MMP. On the other hand, treatment with the combination of DCA+C75 elevated MMP of both the P and N lymphocytes. (FIG. 4E).

### Example E. Lower pHi by Imbalance of ATP Synthesis and Hydrolysis

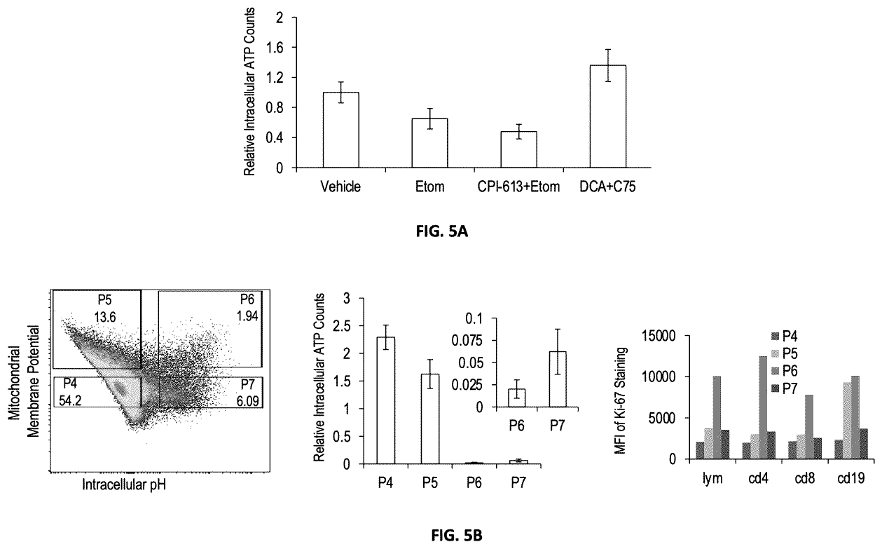

The lymphocytes in the MLN of the asthma model in the late-e2 state were also used to study the relations between intracellular ATP counts and pHi, cell death and proliferation because they comprise diverse subpopulations of lymphocytes including those with high MMP with strong correlation between MMP and pHi and those with low MMP and weak or no correlation between MMP and pHi (the n subpopulation within the N population) (see FIG. 1C). The lymphocytes treated with the vehicle, Etom, CPI-613+Etom and DCA+C75 were freeze-thawed to release intracellular ATP, and ATP counts were measured by luciferase activities that are dependent on ATP concentration. The results showed that treatment with Etom or Etom+CPI-613 lowered intracellular ATP whereas DCA+C75 increased intracellular ATP. (FIG. 5A). Thus, the high percentages of early apoptotic cells in the MLNs of the Etom- and (Etom+CPI-613)-treated mice (FIG. 4D) could be due to the reduction of ATP production in those lymphocytes, which would lead to the rate of ATP hydrolysis exceeding that of ATP synthesis thereby lower pHi and induce apoptosis.

Further studies were carried out to probe the intricate relations between intracellular ATP counts and cell death and proliferation. To this end, MLN lymphocytes from mice untreated with the compounds were sorted into 4 populations of P4, P5, P6 and P7 based on their pHi and MMP profiles, (FIG. 5B left), where the P6 population was equivalent to the n subpopulation in FIG. 1C. Despite their low MMP, the P4 lymphocytes had the highest intracellular ATP counts, (FIG. 5B middle), suggesting that the P4 cells May use mechanisms other than mitochondrial respiration, for example, glycolysis, to supplement mitochondrial energy production.

In contrast, the P6 cells, which had MMP similar to that of the P5 cells, had the lowest intracellular ATP counts. (FIG. 5B middle). However, rather counter-intuitively, the P6 cells were the most proliferative as judged by their highest levels of Ki-67 expression. (FIG. 5B right). These results suggest that the P6 cells were prone to apoptosis due to insufficient supply of ATP to power their high rates of proliferation. This would tip the balance between ATP synthesis and hydrolysis to hydrolysis thereby lower pHi. This scenario was further supported by the fact that increase of the influx of pyruvates and fatty acids by the treatment of DCA+C75 treatments increased intracellular ATP (FIG. 5A).

### Example F. Control Lymphocyte Populations in Late-e1 State in Asthma Model

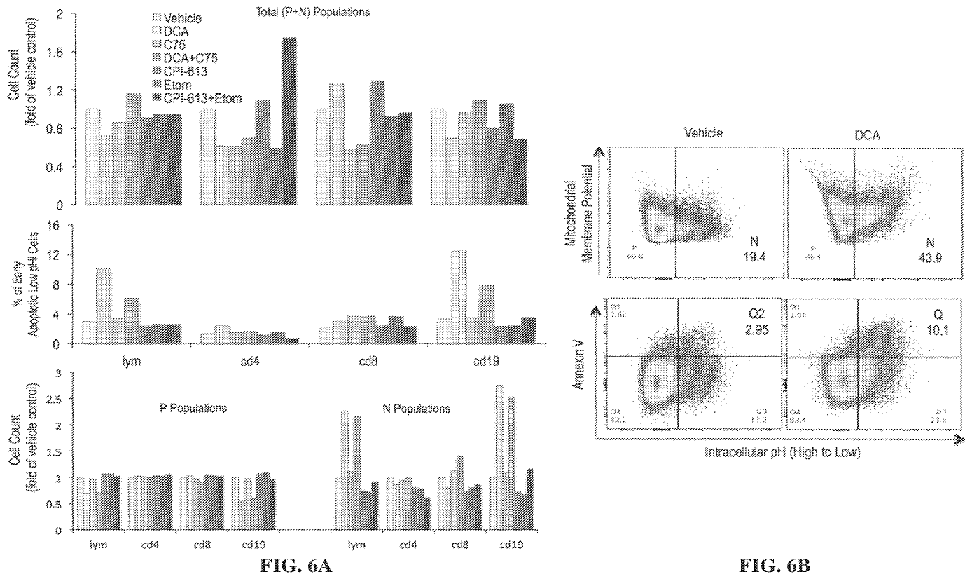

In this example, mice sensitized and challenged with OVA and treated with compounds that either enhance (DCA, C75) influx of pyruvates or fatty acids to TCA cycle or restrict the entry of the carbon backbones of amino acid (CPI-613) or the influx of fatty acids (Etom) to the TCA cycle or with combinations of these compounds. (FIG. 6A). DCA, C75, DCA+C75, and Etom lower total live CD4 T cells in the MLNs. Given that CD4 T cells are the most relevant to the pathogenesis of asthma, this result suggests that these compositions could be used clinically to treat asthma. C75 or C75+DCA decreased total live CD8 T cells, whereas DCA or CPI-613 modestly increased the CD8 T cells. C75, CPI-613 or CPI-613+Etom decreased total live B cells. (FIG. 6A). More details on early apoptosis and different effects on P and N lymphocytes were also analyzed and data were presented in FIG. 6A. The N lymphocytes in the e1 state were highly responsive to DCA treatment that increased MMP and early apoptotic cells. (FIG. 6B).

### Example G. Control of Lymphocyte Populations in Late-e3 State in Asthma Model

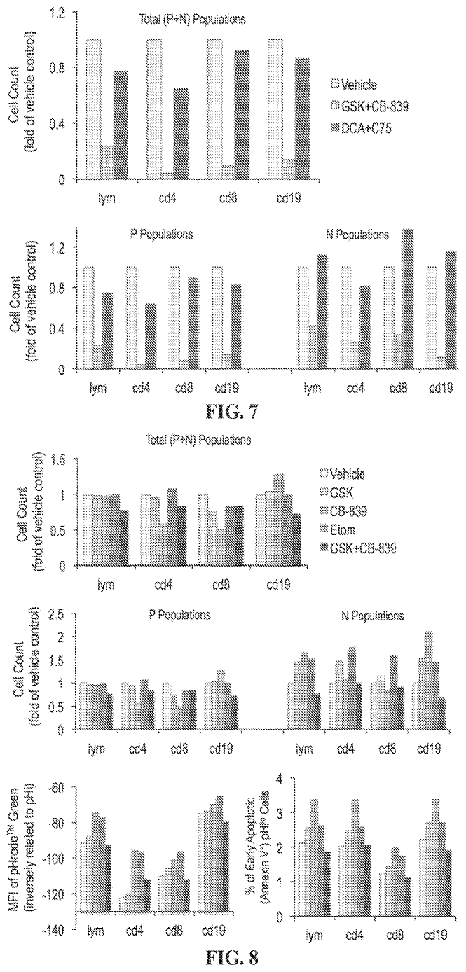

Similar to that in the late-e1 state but with further decline of potency, DCA+C75 caused modest reduction of total live CD4, CD8 T cells and B cells, with the strongest effect on CD4 T cells. (FIG. 7). In contrast, treatment with the combination of GSK+CB-839 that inhibits glycolysis and glutaminolysis reduced total live CD4 and CD8 T cells to less than 5% and 10% of the vehicle control, respectively, and B cells to less than 14%. (FIG. 7).

### Example H. Control of Lymphocyte Populations in Late-e4 State in Asthma Mode

In late-e4 state, lymphocytes retained significant responsiveness to CB-839 but only mild responsiveness to GSK treatment. Treatment with CB-839 caused substantial reduction of CD4 and CD8 T cells but not B cells. However, the combination of GSK+CB839 caused modest reduction in all lymphocyte populations. (FIG. 8).

### Example I. Control of Immune Response in Two Rounds of Antigenic Challenges in Asthma Model

In this example, OVA-sensitized mice received the first round of OVA challenges and treatments with regulators of energy metabolism. Twenty-eight days after the first OVA challenge in the first round, the mice received a second round of OVA challenges and treatments with the regulators of energy metabolism. The day the mice received the first OVA challenge in both rounds was defined as the day 0. Three treatment regimens were employed: (a) on days 2, 3 and 4 of the first round, mice received i.p. injection of the combination of CPI-613+Etom, in the second round of challenges, the mice received the same treatment on days 3 and 4; (c) on days 4, 5, 6, 7, 8, 9, 10 in the first round, mice received i.p. injection of a combination of GSK+CB-839, and on day 5 in the second round received the same treatment; (d) on days 2, 3, and 4 in the first round, mice received i.p. injection of a combination of DCA+C75, and on days 4, 5, 6, 7, 8, 9, 10, received i.p. injection of a combination of GSK+CB-839, and in the second round, on days 3 and 4 received i.p. injection of the combination of DCA+C75, and on day 5 received i.p. injection of the combination of GSK+CB-839; in addition a group of mice also received i.p. injection of HOAc on days 2, 4, 6, 8 and 10 in the first round, and days 3 and 4 in the second round. On day 5 in the second round, all compound-treated and vehicle treated mice were assayed for airway hypersensitivity, and MLNs were harvested for immunological analyses.

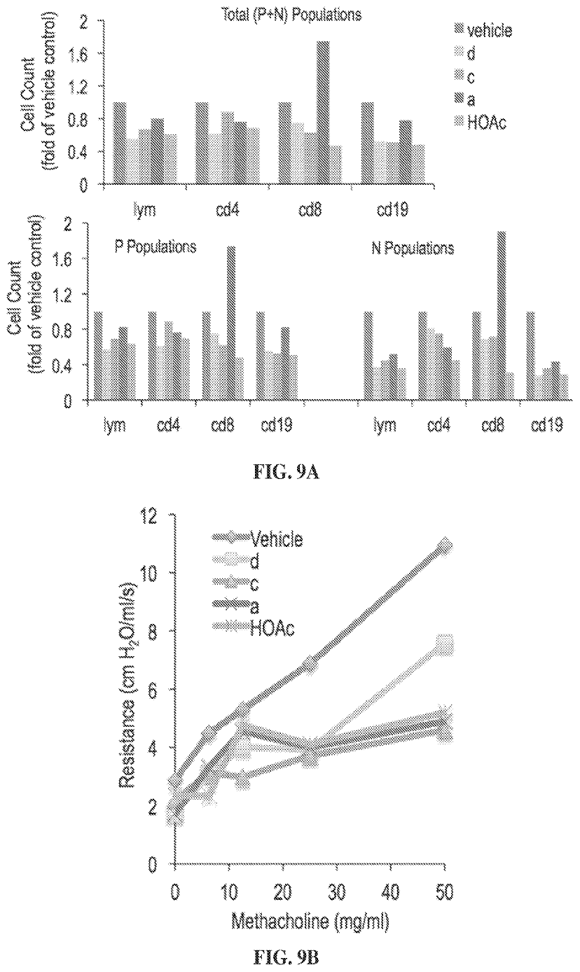

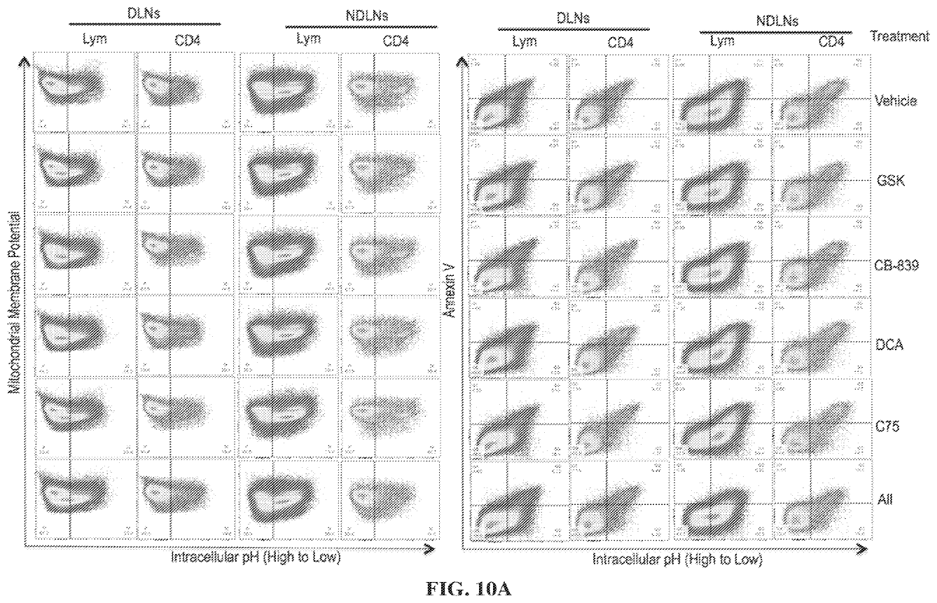

In the MLNs, all treatment regimens reduced CD4 T cells, which are considered the most relevant to asthma pathogenesis, and the B cells. All treatment regimens except regimen (a) also reduced CD8 T cells. (FIG. 9A). In diseases such as asthma, immune response to antigen stimulation is considered the key mechanism of pathogenesis, controlling such immune response is expected to relieve or cure a subject of the clinical symptoms. While it is not possible to measure all clinical symptoms of the disease in a mouse model, as an example, the effects of the treatments on airway hyper-responsiveness (AHR), an important pathophysiological symptom of asthma, were tested. Compared with vehicle control, all treatment regimens reduced AHR in the mice. (FIG. 9B).

### Example J. Control Immune Response in EAE

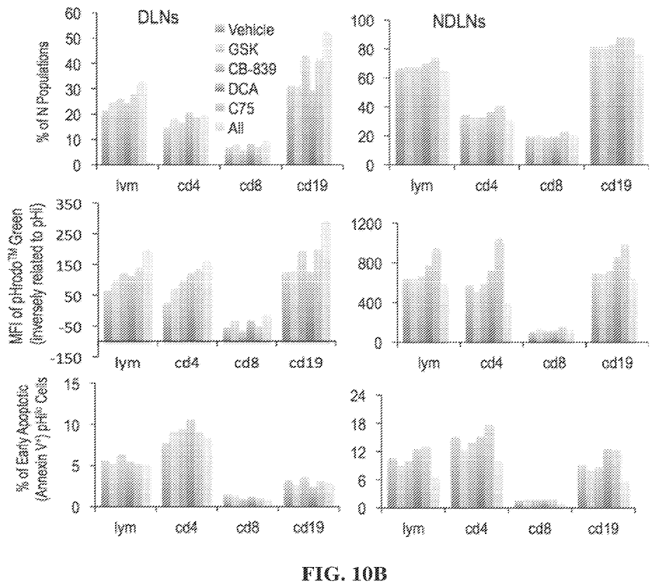

EAE is a mouse model for the human disease multiple sclerosis, and is induced by subcutaneously immunizing mice with the myelin oligodendrocyte glycoprotein (MOG) peptide. It is generally accepted that CD4 T cell response to the MOG peptide is the key mechanism for pathogenesis. In the first set of experiments in this example, the mice were treated with different regulator(s) as shown in FIG. 10A and both the draining lymph nodes (DLNs) and non-draining lymph nodes (NDLNs) from the mice were analyzed. The profiles of pHi and MMP showed that the lymphocytes in the DLNs were in the late-e1 state where substantial N lymphocytes had low MMP and showed a weak inverse relation between MMP and pHi, but there were also some N lymphocytes that had high MMP and strong inverse relation between MMP and pHi. In the NDLNs, the lymphocytes were at the intermediate stage where the N lymphocytes were the dominant population but the P lymphocytes showed a positive relation between MMP and pHi. (FIG. 10A). Treatments with GSK, CB-839, DCA, C75, or a combination of all 4 compounds increased the N populations and lowered pHi in total live lymphocytes and the critical live CD4 T cells. In agreement with the notion lower pHi induces apoptosis, the percentages of early apoptotic cells in the CD4 T cells were also increased in the mice treated with the compound(s). (FIG. 10B). It is also noteworthy that increase of early apoptosis in CD4, CD8 T cells and B cells were not observed in the NDLNs of mice treated with GSK, CB-839 and the combination of all four compounds. (FIG. 10B, bottom right). This finding suggests that such treatments would less likely cause nonspecific depletion of lymphocytes that may compromise a subject's ability to respond to infection. In a separate experiment, mice were treated with vehicle or a combination of GSK and CB-839 and monitored for EAE clinical symptoms. Over a course of about 4 weeks after immunization, 75% of the vehicle control mice developed EAE, whereas none of the mice treated with the combination of GSK+CB-839 developed disease.

### Example K. Combinatorial Approach to Lowering pHi for Cancer Therapy

As described above, study of normal lymphocytes has demonstrated that there can be multiple ways to manipulate energy metabolism to lower pHi to induce apoptosis, namely, by enhancing the influx of pyruvates and/or fatty acids to the TCA cycle; inhibiting amino acid energy metabolism such as glutaminolysis and/or glycolysis; inducing imbalance between ATP synthesis and hydrolysis. Therefore, it was hypothesized that partially targeting multiple pathways of energy metabolism could collectively lower the pHi of tumor cells to a level sufficient to induce apoptosis. Since this strategy does not completely block or overdrive any one of the targeted pathways, it is less likely to cause severe adverse side effects yet in the meantime improve therapeutic efficacy over that targeting only a single pathway. Based on this hypothesis, 9 compositions were designed, comprising multiple compounds, each of which targets a different metabolic pathway that was demonstrated herein to alter pHi. These compositions along with compositions of their individual components are coded by numbers and summarized in Table 1. Assuming the total volume of blood in a mouse is about 2 ml, (30,31), when injected into a mouse the maximum blood concentration of individual compound would not exceed the reported IC50. For compositions marked by a 27 single prime (′), the maximum blood concentration of one of this component is expected not to exceed 1/10 of the IC50.

### Table 1. Compositions of Regulators of Energy Metabolism

The concentrations in this table are concentrations of the compounds in saline. For injection to mice, 200 microliters of a composition per mouse is used; for cell cultures, the composition was added to medium at 1:10 dilution.

| | | Action | Function | | | | | | | |
|---|---|---|---|---|---|---|---|---|---|---|
| Component | | on target | in energy | Composition Code | | | | | | |
| code | Component | enzyme | production | 1 | 3′ | 4 | 5 | 13′ | 14′ | 15 |
| 1 | GSK2837808A | Lactate | Glycolysis | 52.8 | | | | | | |
| | | dehydrogenase | inhibitor | nM | | | | | | |
| | | inhibitor | | | | | | | | |
| 3′ | Aminooxyacetic | Malate- | Malate- | | 0.11 | | | | | |
| | acid (AOA) | aspartate | aspartate | | mM | | | | | |
| | | shuttle | shuttle | | | | | | | |
| | | inhibitor | inhibitor | | | | | | | |
| 4 | CB- | Glutaminase | Glutaminolysis | | | 11 | | | | |
| | 839 | inhibitor | inhibitor | | | μM | | | | |
| 5 | Sodium | Pyruvate | Promoter of | | | | 11.01 | | | |
| | dichloroacetate | dehydrogenase | influx of | | | | mM | | | |
| | (DCA) | kinase | pyruvates | | | | | | | |
| | | inhibitor | to TCA | | | | | | | |
| | | | cycle | | | | | | | |
| 13′ | CPI- | pyruvate | Inhibitor | | | | | 0.11 | | |
| | 613 | dehydrogenase, | of entry | | | | | mM | | |
| | | a- | of amino | | | | | | | |
| | | ketoglutarate | acid carbon | | | | | | | |
| | | dehydrogenase | backbones | | | | | | | |
| | | inhibitor | to TCA | | | | | | | |
| | | | cycle | | | | | | | |
| 14′ | C75 | Carnitine | Promoter of | | | | | | 0.11 | |
| | | palmitoyl | influx of | | | | | | mM | |
| | | transferase | fatty | | | | | | | |
| | | 1 (CPT-1) | acids to | | | | | | | |
| | | activator | TCA | | | | | | | |
| | | | cycle | | | | | | | |
| 15 | Etomoxir | Carnitine | Inhibitor | | | | | | | 0.55 |
| | | palmitoyl | of influx | | | | | | | mM |
| | | transferase | of fatty | | | | | | | |
| | | 1 (CPT-1) | acids to | | | | | | | |
| | | inhibitor | TCA | | | | | | | |
| | | | cycle | | | | | | | |

| Component | Composition Code | | | | | | | | | |
|---|---|---|---|---|---|---|---|---|---|---|
| code | 8′ | 18′ | 19 | 11 | 12′ | 16′ | 20′ | 21′ | 17′ | 22 |
| 1 | 52.8 | 52.8 | 52.8 | 52.8 | 52.8 | 52.8 | 52.8 | 52.8 | | |
| | nM | nM | nM | nM | nM | nM | nM | nM | | |
| 3′ | 0.11 | | | | 0.11 | | | 0.11 | | |
| | mM | | | | mM | | | mM | | |
| 4 | 11 | 11 | 11 | 11 | 11 | 11 | 11 | 11 | | |
| | μM | μM | μM | μM | μM | μM | μM | μM | | |
| 5 | | | | 11.01 | 11.01 | 11.01 | | 11.01 | 11.01 | |
| | | | | mM | mM | mM | | mM | mM | |
| 13′ | | 0.11 | | | | | | | | 0.11 |
| | | mM | | | | | | | | mM |
| 14′ | | | | | | 0.11 | 0.11 | 0.11 | 0.11 | |
| | | | | | | mM | mM | mM | mM | |
| 15 | | | 0.55 | | | | | | | 0.55 |
| | | | mM | | | | | | | mM |

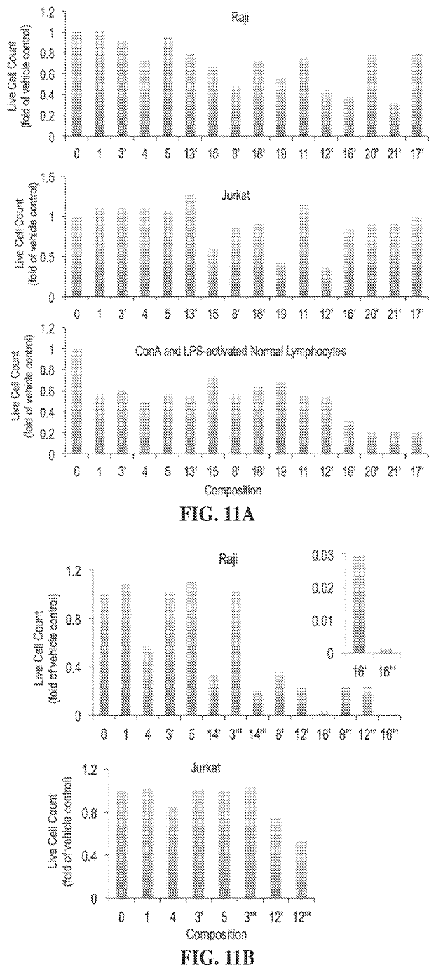

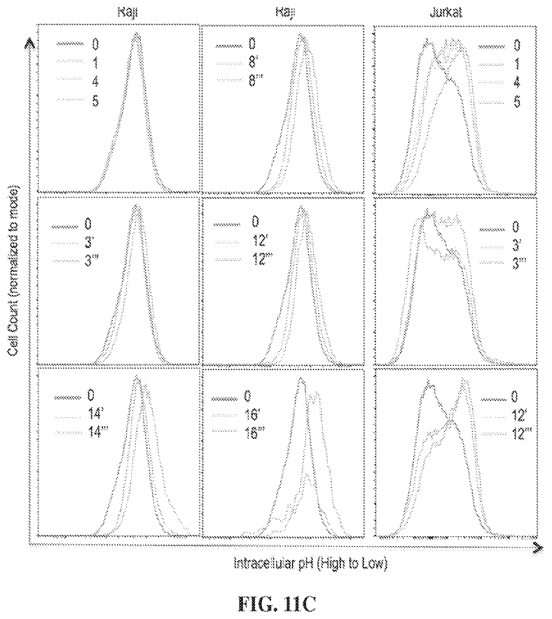

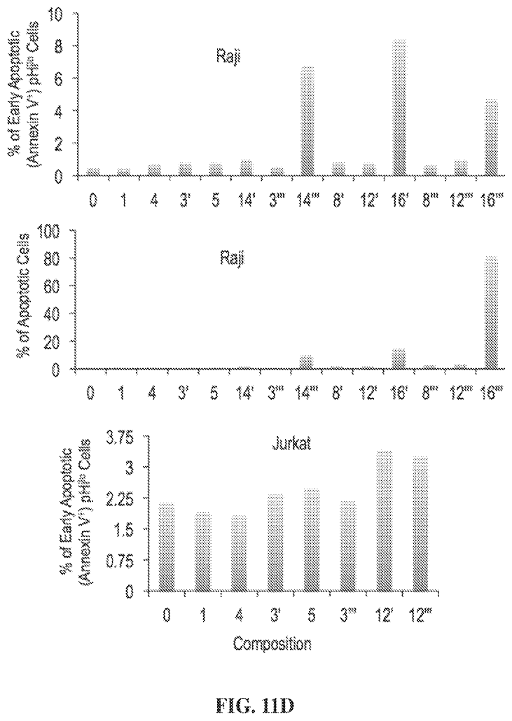

These combinatorial compositions and compositions of their individual components were screened 2 for their abilities to inhibit the growth of the human tumor cell lines Raji and Jurkat in culture. Raji is a B cell lymphoma line, and Jurkat is a T cell leukemia cell line. The compositions were also tested for their inhibitory effects on the growth of normal lymphocytes activated by Concanavalin A (ConA) and lipopolysaccharide (LPS), which serves as a proximate indicator of potential adverse effect of the treatments on the ability of a subject to mount immune response to infection. After cultured with the compositions or vehicle control, the tumor cells or lymphocytes were counted by flow cytometry using 7AAD to distinguish live and dead cells. The result of the first screening showed that compositions 8′, 19, 12′, 16′ and 21′ caused strong inhibition of Raji cell growth, whereas compositions 15, 8′, 19 and 12′ caused strong inhibition of Jurkat cells. In comparison, the individual components of these combinatorial compositions showed no or only small inhibition of the tumor cells. For the in vitro activated normal lymphocytes, compositions 20′ and 21′ were found to cause strong inhibition, indicating that these two compositions may cause potential adverse side effects. (FIG. 11A).

Once the first screening result is available, the concentrations or doses of one or more components of an effective composition may be adjusted to improve its efficacy. As examples of this strategy, combinatory compositions 8′, 12′ and 16′ for Raji and combinatorial composition 12′ for Jurkat were further studied. As described in Table 1, the concentrations of the components that were expected not to reach maximum blood concentration more than 1/10 of IC50 (coded with a number and single prime) were increased to 3 fold of those in the original compositions, and this alteration was indicated by a triple prime (′″). The result showed that the increase of the concentration of a single component in the composition enhanced the potency of inhibition in all combinatorial compositions. In the case of composition 16′″, only scant number of Raji cells survived the treatment. Again, the individual components in these compositions alone showed much less or no inhibition of tumor cell growth. (FIG. 11B).

To demonstrate that the inhibition of tumor growth is associated with lowering pHi and inducing apoptosis, pHi of the tumor cells after treatments with the compositions was analyzed. In most cases, compositions comprising a single component except composition 14′ and 14′″ caused no or small decrease of pHi in Raji cells whereas the reduction was greatly enhanced with the combinatorial compositions. In Jurkat cells the same enhancement was also observed. (FIG. 11C). Early apoptosis of the treated tumor cells were also analyzed. As alluded to before, early apoptosis or apoptosis assays tend to underestimate the effect of a treatment on cell death because dead may disintegrate during in vitro experiment. Nonetheless, increase of early apoptosis of Raji cells by 16′ over those by treatments with single component was detected. However, inhibition of Raji cells by 16′″ was particularly underestimated by the early apoptosis assay, which was less than that of 16′ or 14′″, because there were much fewer live Raji cells and more apoptotic cells after treatment with 16′″ than after 16′ or 14′″.

### Example L. Ki-67 Level Correlates with Susceptibility to Low pHi-Induced Death

Biomarker with predictive value for responsiveness to therapeutics remains elusive for most cancers. Ki-67 is a reliable marker for proliferating cells whose level of expression positively correlates with rRNA and DNA synthesis, but is not a clear predictor of responsiveness to existing chemotherapeutics. In contrary to such previous notion, this example shows that the level Ki-67 is in fact a clear predictor of responsiveness of Raji cells to low pHi.

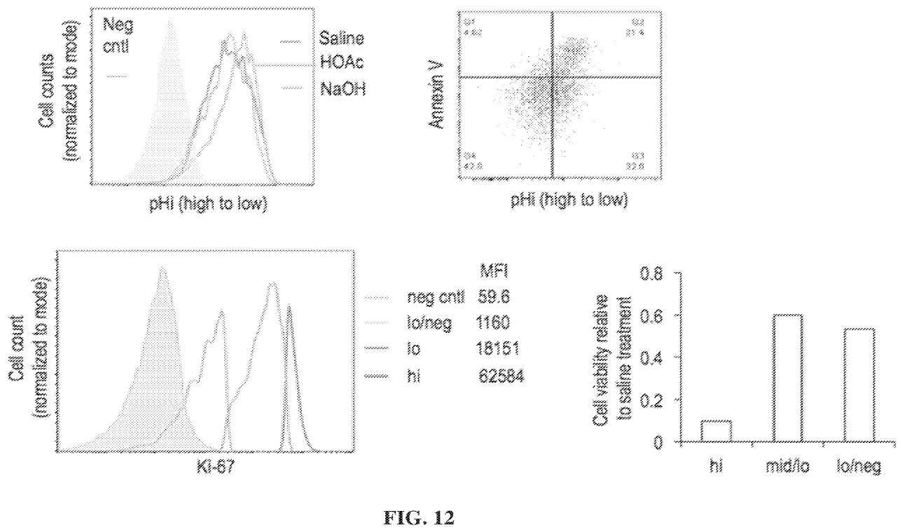

It was found that treatment of Raji cells with HOAc lowered the pHi of the tumor cells, (FIG. 12, upper left), and low pHi in turn induced apoptosis, (FIG. 12, upper right). In further study, Raji cells with high, middle or low, or nearly negative (lo/neg) staining of Ki-67, (FIG. 12, lower left), were treated with saline or saline plus HOAc. Raji cells with high levels of Ki-67 that survived the HOAc treatment were only about 10% of live Raji cells treated with saline. In contrast, Raji cells with middle/low or nearly negative Ki-67 expression that survived the HOAc treatment were about 60% of those after saline treatment. (FIG. 12, lower right). Thus, high level of Ki-67 is potentially an excellent predictive biomarker for low pHi-based cancer therapy.

### Example M. Low pH-based Treatment of Infections of Skin and Mucosa

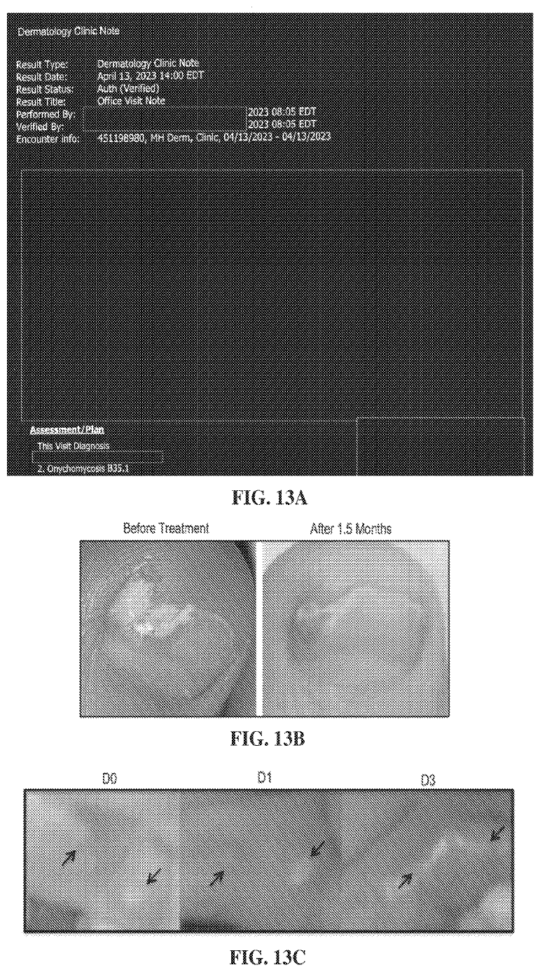

A subject suffered fungal toe infection on the right great toe, which was diagnosed as onychomycosis. (FIG. 13A). The infection was first treated with over-the-counter medicine without improvement, then treated with the prescription drug ketoconazole 2% cream daily for 1.5 month again without noticeable improvement. The ketoconazole treatment was then discontinued, and the subject started to self treat the lesion with topical application of HOAc water solution on the infected nail and surrounding areas. After polishing the nail to remove excess nail mass in the infected area, 12% HOAc was applied daily once in the morning, and once in the evening before bedtime for the first 3 days. Afterwards, 6% HOAc was applied once in the morning and 3% HOAc was applied once before bedtime daily. After about 2 weeks of treatment, great improvement was observed, nonetheless, the treatment continued for a total of 1.5 months. This regimen stopped the fungal growth and greatly improved the appearance of the nail. (FIG. 13B). For treatment of mucosa infection, the subject had two lesions of infection on the inner side of the upper lip caused by accidental biting. The subject self treated the lesions by topical application of a 5% HOAc water solution 4 times a day (approximately every 4 hours). After two days of treatment, both lesions had resolved. (FIG. 13C).

### Example N. Treatment of Skin Neoplasia with pH Modifier

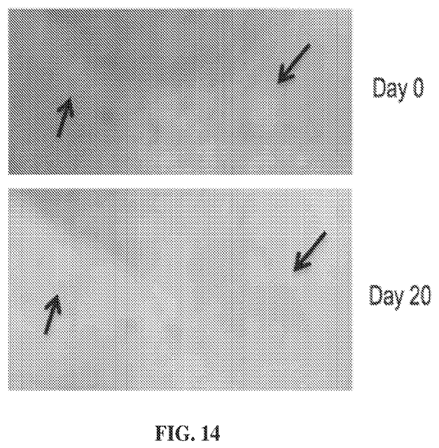

Two blemishes on the face of a subject were clinically diagnosed as benign neoplasia. The subject self treated the neoplastic lesions by topical application of a 12.5% HOAc water solution 3 times (morning, noon, and bedtime) a day for 3 days, followed by topical application of 5% HOAc water solution 3 times (morning, noon, and bedtime) a day for 17 days. Both neoplastic lesions became negligible at the end of the treatments. (FIG. 14).

### REFERENCES

1. p 447, Janeway's Immunobiology, 8th ed. Garland Science, New York.
2. Biochem Biophys Res Commun 313:459-65.
3. Semin Cancer Biol 15:300-8.
4. Medicina (Kaunas) 58.
5. Cancer Res 73:1524-35.
6. Front Immunol 8:184.
7. Eur J Immunol 31:2829-38.
8. Nature 513:559-63.
9. J Immunother 23:196-207.
10. Blood 53:1133-9.
11. p 621-623, Lehninger Principles of Biochemistry. W.H. Freeman and Company, New York.
12. Proc Natl Acad Sci USA 104:19345-50.
13. Biochem Biophys Res Commun 186:782-9.
14. Proc Natl Acad Sci USA 93:654-8.
15. J Biol Chem 275:9244-50.
16. Nat Cell Biol 2:318-25.
17. Nat Rev Compound Discov 21:141-162.
18. Cell 163:712-23.
19. Front Oncol 12:960317.
20. Oncotarget 6:11735-50.
21. Curr Opin Immunol 14:354-9.
22. Cell Death Differ 25:46-55.
23. Nature 261:702-705.
24. Immunol Rev 249:43-58.
25. Cell 168:960-976.
26. J Allergy Clin Immunol 139:508-518 e4.
27. Lab Anim 21:31-4.
28. J Immunol 183:3583-90.
29. Annu Rev Immunol 36:489-517.
30. p 372. In Harkness J, Wagner J (ed), The biology and medicine of rabbits and rodents, 3^{rd} ed. Lea & Febiger, Philadelphia.
30. Mitruka B, Rawnsley H. 1981. Clinical, biochemical and hematological reference values in normal experimental animals and normal humans. Masson Publishing, New York.

1.-15. (canceled)

16. A composition for inducing apoptosis by lowering intracellular pH (pHi) or promoting cell survival and proliferation by raising pHi, by modulating energy metabolism of the cell for disease treatment or prevention, wherein the diseases comprise but are not limited to immunological diseases and neoplastic diseases and the cells comprise but are not limited to lymphocytes and cancer cells, and whereby,
lowering pHi is achieved by increasing the influx of pyruvates and/or fatty acids to the TCA cycle, inhibiting amino acid energy metabolism, inhibiting glycolysis for energy production, inducing imbalance between ATP synthesis and hydrolysis by restricting entry of carbon backbones of amino acids and/or influx of fatty acids to TCA cycle, or combinations thereof;
raising pHi is achieved by enhancing amino acid energy metabolism and/or glycolysis for energy production, restricting influx of pyruvates and/or fatty acids to the TCA cycle, restoring balance between ATP synthesis and hydrolysis, or combinations thereof; and
modulating energy metabolism is achieved by administering to a subject the composition comprising one or more of regulator(s) of energy metabolism; wherein the regulators of energy metabolism that lowers pHi comprise but are not limited to:
sodium dichloroacetate (DCA) and/or other compounds that increase the activity and/or expression of pyruvate dehydrogenase, which increases the influx of pyruvate to the TCA cycle;
C75 and/or other compounds that increase the activity and/or expression of carnitine palmitoyltransferase 1 (CPT-1), which increases the influx of fatty acids to the TCA cycle;
GSK2837808A and/or other compounds that inhibit the activity and/or expression of lactate dehydrogenase, which inhibits glycolysis;
CB-839 and/or other compounds that inhibits glutaminolysis, and/or other compounds that inhibit energy metabolism of other amino acids;
CPI-613 and/or other compounds that inhibits the entry of carbon backbone of amino acids to the TCA cycle, and/or Etomoxir and/or other compounds that inhibit the influx of fatty acids to the TCA cycle, which induce imbalance between ATP synthesis and hydrolysis; and
the composition for lowering pHi comprises one of the afore said regulators or the combination of DCA, C75, GSK2837808A and CB-839 or one of any other combinations of the afore said regulators;
and conversely, the regulators of energy metabolism that raises pHi comprise but are not limited to:
compounds that decrease the activity and/or expression of pyruvate dehydrogenase, which decreases the influx of pyruvate to the TCA cycle;
compounds that decrease the activity and/or expression of carnitine palmitoyltransferase 1 (CPT-1), which decreases the influx of fatty acids to the TCA cycle;
compounds that increase the activity and/or expression of lactate dehydrogenase, which increases glycolysis;
compounds that increase glutaminolysis, and/or other compounds that increase energy metabolism of other amino acids;
compounds that increase the entry of carbon backbones of amino acids to the TCA cycle, and/or compounds that increase the influx of fatty acids to the TCA cycle, which restore/maintain the balance between ATP synthesis and hydrolysis;
and the composition for raising pHi comprises one or more of the afore said regulators.

17. Use of the composition of claim 16 in the manufacture of a medicament for disease treatment and/or prevention, wherein the composition may comprise one or more regulators of energy metabolism, and wherein the combinatorial composition comprises low doses or concentrations of individual regulators so that collectively they can effectively alter pHi but avoid severe adverse side effects.

18. The composition of claim 16, wherein the disease is an immunological disease, and the composition is for controlling immune response via targeting mechanisms of energy metabolism that impact the pHi of lymphocytes and operate in the lymphocytes at different stages in the natural time course of the immune response, wherein the targeting is guided and the natural time course is determined by the profiles of intracellular pH (pHi) and mitochondrial membrane potentials (MMPs) of the lymphocytes.

19. Use of reagents and chemicals to manufacture a kit for guiding the use of the composition of claim 18 by determining the pHi and MMPs of the lymphocytes or the stages in the natural time course of an immune response; wherein
the said stages are determined by dividing the lymphocytes into populations of relatively high and low pHi that are referred to as the P and N lymphocytes or populations, respectively, and further by the profiles of both the pHi and MMPs of the P and N lymphocytes such that
the early stage is determined by a predominance of N lymphocytes, and all lymphocytes show an inverse relation between MMP and pHi;
the intermediate stage is determined by the appearance of P lymphocytes with a positive relation between MMP and pHi and the N lymphocytes may or may not remain as the dominant lymphocytes;
the late stage is determined by a predominance of P lymphocytes with a positive relation between MMP and pHi;
alternatively, the criteria for determining the stages in the natural time course of an immune response are the differential patterns of molecules and/or the activities of the molecules that are present or expressed in the lymphocytes at the different stages as determined by the foregoing method; and wherein
the reagents and chemicals comprise but are not limited to pHrodo™ dyes and/or other pHi indicators, MitoSpy dyes and/or other MMP indicators, and those for detecting the differential patterns of molecules and/or the activities of the molecules that are present or expressed in the lymphocytes at the different stages in the natural time course of immune response.

20. The composition of claim 16 wherein the guidance for its use comprises a laboratory technique to stain cells for surface molecule(s), pHi, MMP and Annexin V in a single step.

21. The composition of claim 16, wherein the immunological diseases are those with pathogenesis attributed partially or fully to overzealous immune response comprising but not limited to autoimmune diseases, allergic diseases, and infectious diseases where immune responses impair the structure and/or functions of host tissue or organ, wherein
the composition for treatment and/or prevention is a composition for lowering pHi of lymphocytes to induce apoptosis according to the natural time course of the immune response or the profiles of pHi and MMPs of the lymphocytes or the relative proportions of P and N lymphocytes, wherein,
at the early stage primarily by increasing the influx of pyruvates and/or fatty acids to the TCA cycle, and optionally by inhibiting amino acid energy metabolism and/or glycolysis for energy metabolism, and/or inducing imbalance between ATP synthesis and hydrolysis by restricting entry of carbon backbones of amino acids and/or influx of fatty acids to TCA cycle, or combinations thereof;
at the intermediate stage primarily by inhibiting amino acid energy metabolism, increasing the influx of pyruvates and/or fatty acids to the TCA cycle, and optionally by inhibiting glycolysis for energy production, inducing imbalance between ATP synthesis and hydrolysis by restricting entry of carbon backbones of amino acids and/or influx of fatty acids to TCA cycle, or combinations thereof;
at the late stage in the natural time course of immune response primarily by inducing imbalance between ATP synthesis and hydrolysis by restricting entry of carbon backbones of amino acids and/or influx of fatty acids to TCA cycle, inhibiting glycolysis for energy production and/or amino acid energy metabolism, and optionally by increasing influx of pyruvates and/or fatty acids to TCA cycle, or combinations thereof.

22. The composition of claim 16, wherein the immunological diseases are diseases whose pathogenesis is partially or fully attributed to insufficient immune response to infection, vaccine or cancer, and wherein the composition for the disease treatment or prevention is the composition for raising pHi of lymphocytes to promote survival and proliferation of the lymphocytes.

23. The composition of claim 16, wherein the disease is a neoplastic disease, and the composition for lowering pHi is designed in a combinatorial manner to induce apoptosis of neoplastic cells by:
creating a panel of compositions of individual regulators of energy metabolism at low doses or concentrations and combinations of the low doses/concentrations of the individual regulators to lower pHi of the neoplastic cells; and
screening such panel of compositions for inhibition of the growth and/or survival of the neoplastic cells; and with or without simultaneously
screening such panel of compositions for inhibition of the growth or survival of normal cells,
selecting composition(s) that show relatively strong inhibition of neoplastic cells, with or without also considering relatively weak or no inhibition of normal cells,
further adjusting the dose(s) of one or more regulator(s) of energy metabolism in the selected compositions to achieve optimal inhibition of neoplastic cells but minimize inhibition of normal cells.

24. Use of the composition of claim 23 in the manufacture of medicaments for treating a neoplastic disease, wherein the composition is one or more of compositions in the panel of compositions.

25. The composition of claim 16 comprise compositions summarized in Table 1 and/or described in Example K in this application and compositions of metabolic regulators with similar or identical functions; wherein the compositions comprise but are not limited to compositions formulated identically or similarly to with energy metabolic regulator(s) identical or similar to those in 8′, 12′, 15, 16′, 19, 21′, 8′″, 12′″ and 16′″ described in Table 1 and/or Example K are combinatorial anti-cancer drugs.

26. Use of reagents and chemicals to manufacture a kit to detect Ki-67 to improve therapeutic outcome of low-pHi based cancer therapy by using Ki-67 as a biomarker to predict a subject's responsiveness to low pHi-based cancer therapy wherein the level of Ki-67 expression in cancer cells or the percentage of cancer cells expressing high level(s) of Ki-67 serves as a predictor for the subject's responsiveness to the low pHi-based cancer therapy; wherein
The low pHi-based therapy is by administering to a subject a composition comprising pH modifier(s) that lower pHi of cancer cells, and/or composition of claim 16.

27. A composition for treating skin or mucosa infection(s) or neoplasia by topical application of pH modifier(s) and/or composition of claim 16 that lower pH.

28. Use of the composition of claim 27 in the manufacture of a medicament for treating skin or mucosa infection(s) or neoplasia.
